(19) United States
(12) Patent Application Publication
(10) Pub. No.: US 2008/0311189 A1
(43) Pub. Date: Dec. 18, 2008

(54) Compositions Comprising Organometallic Molybdenum Compounds For Treating Cancer

(76) Inventors: Marta R.P. Norton Matos, Lisbon (PT); Carlos C. Romao, Cascais (PT); Claudia Cristina Lage Pereira, Oeiras (PT); Sandra S. Rodrigues, Moscavide (PT); Marcia Mora, Cacem (PT); Maria Joao Pires Silva, Odivelas (PT); Paula M. Alves, Oeiras (PT); Celso Albuquerque Reis, Vila Nova De Gaia (PT)

Correspondence Address: SUGHRUE MION, PLLC, 2100 PENNSYLVANIA AVENUE, N.W., SUITE 800, WASHINGTON, DC, 20037, US

(21) Appl. No.: 10/593,283

(22) PCT Filed: Mar. 18, 2004
(86) PCT No.: PCT/PT2004/000004

§ 371 Date: May 16, 2008

(51) Int. Cl. A61K 9/48; A61K 31/28; A61K 31/66

(52) U.S. Cl. 424/456; 514/188; 514/492; 514/185; 514/187; 514/107

(57) ABSTRACT

The invention provides several molybdenum (II) complexes (see classes I and II, FIG. 1) as well as pharmaceutical compositions comprising these compounds, that are useful for treating cancer and describes synthetic methods and intermediates useful for preparing the compounds.

### FIELD OF INVENTION

The present invention describes organometallic molybdenum (II) complexes and pharmaceutical compositions containing said complexes, effective for treating cancer cells, in particular the Ehrlich-ascites mouse cancer cells and the human gastric and colon cancer cells.

### BACKGROUND OF THE INVENTION

Cancer diseases are together with angiocardiopathies the main causes of death in most developed countries. Although cancer is often referred to as a single condition, it actually consists of more than 100 different diseases, all characterized by the uncontrolled growth and spread of abnormal cells. Basic and applied research into the causes and cures for cancer continues, including investigations designed to change screening, diagnosis, and treatment. In principle, cancer diseases can be treated by surgery, radiation and chemotherapy.

Cancer chemotherapy kills or arrests the growth of cancer cells by targeting specific parts of the cell growth cycle. However, normal healthy cells share some of these pathways and are also injured or killed by chemotherapy. In particular rapidly growing cells—including blood cells and epithelial cells, in particular in hair follicles and in the gastrointestinal tract—are most likely to be damaged causing severe side effects. The main challenge in cancer chemotherapy today is the discovery and development of new molecules which selectively injure/kill tumor cells without affecting normal cells. In principle this can be achieved through the identification of targets specific to the function of tumor cells. Recently few such targets have been identified but no new anti-cancer drugs have yet been developed.

The history of a systematic therapy of cancer using medicines started only about sixty years ago. Until the middle of the 1970's, organic compounds such as alkylating agents, antimetabolites and vinca rosea alkaloids were the most common cytostatic drugs, generally administred as drug combinations with or without surgery and/or radiation. (Köpf-Maier, P.; Köpf, H. Structure and Bonding, 1988, 70, 105-185). Towards the end of the 1970's a newly developed inorganic platinum complex, cis-(NH₃)₂PtCl₂, was introduced into clinical use and added to the panel of approved cytostatics. Cisplatin is one of the most effective antitumor agents. It is unique in that it is capable of curing most patients suffering from testicular carcinomas. Cisplatin also prolongs the survival of many patients suffering from ovarian, bladder, prostate lung, head and neck carcinomas. Today, cisplatin and its second generation analog carboplatin, are the most frequently applied cytostatic drugs (Harrap, K. R. Cancer Tret. Rev. 1985, supl. A, 21-33).

This clinical success together with the need to overcome the resistance and toxicity of Pt(II) compounds stimulated a broad search for other metal-containing anti-cancer drugs. Various and structurally different types of non-platinum metal complexes have been screened either in vitro or in vivo and have been found to be effective against experimental tumours in animals. These compounds comprise main-group metallic compounds of gallium, germanium, tin and bismuth, early-transition metal complexes of titanium, vanadium, niobium, molybdenum and rhenium, and late-transition metal complexes of ruthenium, rhodium, iridium, platinum, copper and gold (Keppler, B. Metal complexes in cancer chemotherapy, VCH: Basel, 1993).

In 1979 Köpf and Köpf-Maier reported the antitumor activity of an extensive range of neutral metallocene dihalides and diacido complexes Cp₂MX₂ (Cp=C₅H₅); M=Ti, V, Nb, Mo, Re; X=halide or diacido ligand) against mouse tumor models and several human tumors xenografted into athymic mice (Köpf-Maier, P.; Köpf, H. U.S. Pat. No. 4,608,387, Aug. 26, 1986). The leading compound in this class of compounds is titanocene dichloride, Cp₂TiCl₂, currently under phase II clinical trials. Further results are required to establish whether this complex will become a clinically useful drug for treating cancer. Its poor solubility and stability at pH 6-7 are the main drawbacks for the development of, a suitable formulation (Harding, M. M.; Mokdsi, G., Curr. Med. Chem. 2000, 7, 1289-1303).

Another group of antitumor neutral metallocenes derivatives are the uncharged decasubstitued metallocenes with the main group elements tin (II) or germanium (II) as central metals in the +2 oxidation state (Köpf-Maier, P.; Janiak, C.; Schumann, H. J. Cancer Res. Clin. Oncol. 1988, 114, 502-506).

Antitumor activity is not confined to neutral metallocenes but is also found for ionic derivatives. This was shown for ionic titanocene (Köpf-Maier, P.; Neuse, E.; Klapötke, T.; Köpf, H. Cancer Chemother. Pharmacol. 1989, 24, 23-27), rhenocene (Köpf-Maier, P.; Klapötke, T. Cancer Chemother. Pharmacol. 1992, 29, 361-366) and the highly oxidized niobocene and molybdenocene complexes (Köpf-Maier, P.; Klapötke, T. J. Cancer Res. Clin. Oncol. 1992, 118, 216-221).

Besides these ionic metallocenes, Köpf also reported on the antitumor activity of diverse ferrocenium complexes [(Cp)₂Fe]⁺X⁻ with X=SbCl₆, 2,4,6-(NO₂)₃C₆H₂O or CCl₃CO₂.CCl₃CO₂H (Köpf-Maier, P.; Köpf, H.; Neuse, E. W. J. Cancer Res. Clin. Oncol., 1984, 108, 336-340).

Recently there has been a renewed interest in the anti-tumoral properties of vanadocenes and other vanadium related complexes. Studies conducted at the Parker Hughes Institute (Ghosh, P.; D'Cruz O. J.; Narla, R. K.; Uckun, F. M. Clin. Cancer Res. 2000, 6, 4, 1536-45) investigated the antitumoral activity of 19 vanadocene complexes for treating testicular cancer. These compounds were tested against the human testicular cancer cell lines Tera-2 and Ntera-2 and exhibited significant cytotoxicity inducing apoptosis within 24 hours. Vanadocenes with dithiocyanate [Cp₂V(SCN)₂] and diselenocyanate [Cp₂V(NCSe)₂] as ancillary ligands were identified as the most potent cytotoxic compounds.

In a continuing effort to develop drugs with a broader spectrum of anti-tumoral activity, the same researchers (Narla R. K.; Dong, Y.; D'Cruz, O. J.; Navara, C.; Uckun, F. M. Clin.l Cancer Res., 2000, 6, 1546-1556) synthesized 15 oxovanadium(IV) complexes and examined their cytotoxic activity against 14 different human cancer cell lines. The results obtained showed that oxovanadium compounds induce apoptosis in human cancer cells and may be useful for treating cancer. These drugs are now being tested in animal safety studies to identify those that have the best therapeutic index.

Besides the above mentioned neutral and ionic molibdenocenes complexes other molybdenum containing molecules have been described to display cancerostatic activity:

Na₂MoO₄ was shown to significantly inhibit the incidence of esophagus and forestomach cancers induced by N-nitrososarcosine ethyl ester in Sprague-Dawley (SD) rats. (Luo, X. M.; Wei, H. J.; Yang, S. P. J. Natl. Cancer Inst., 1983, 71, 75).

Molybdenum alone was demonstrated to exert an inhibiting effect on the mammary carcinogenesis in SD rats produced by intravenous injections with nitrosomethylurea (H. Wei, X. Luo, and X. Yang, Chem. Abstr., 1988, 108, 1995).

Heteropolyacid salts of molybdenum and tungsten were described as new cancerostatic drugs, manifesting notable efficacy for solid tumors (European patent, 1988, application number 88905227.0).

In 1992, Fujita et al. (Fujita, H.; Fujita, T.; Sakurai, T.; Yamase, T.; Seto, Y. Tohoku J. Exp. Med., 1992, 168, 421-426) reported the anti-tumoral properties of polyoximolybdates with structures based on closely packed oxygen arrays containing interstitial metal centers. Some of these compounds suppressed the growth of Co-4 human colon cancer xenografted in athymic mice. Potent antitumor activity was also observed against MX-1 human breast and OAT human lung cancer xenografted in athymic nude mice.

In 2000, Hall et al. (Hall, I. H.; Lackey, C. B.; Kistler T. D.; Durham, R. W.; Russell, J. M., Grimes, R. N. Anticancer Res. 2000, 20, 4245-4254) showed that molybdenum complexes that are bound to small carborane ligands C₂B₄ or C₂B₃ exhibit strong cytotoxic effects in murine and human cultured cells, being more effective against suspended leukemia and lymphomas but surprisingly also against selected solid tumors.

In 2001 Xiaoming, L. et al. disclosed the synthesis, and anti-tumoral activity of chiral octahedral molybdenum and tungsten complexes (Shuncheng, L.; Xiaoming, L.; Jingrong, C. patent number CN1321644, Nov. 14, 2001).

Another approach in treating cancer is to prevent the formation of new blood vessels (angiogenesis) that are required for the tumors to grow as their nutritional needs increase. In this respect, tetrathiomolybdate has been found to be an effective antiangiogenic agent by chelating to copper which is an essential cofactor for the building of new blood vessels in tumors (Brewer, G. J.; Dick, R. D.; Grover, D. K.; Le Claire V.; Tseng, M.; Wicha, M.; Pienta, K.; Redman, B. G.; Jahan, T., Sondak, V. K.; Strawderman, M.; LeCarpentier, G.; Merajver, S. D. Clin. Cancer Res. 2000, 6, 1-10). Tetrathiomolybdate lowers the body's copper level into a well-defined but apparently not too narrow “window” of mild copper deficiency, where angiogenesis is brought to a halt without any other major side effects. Ongoing phase II clinical trials evaluated the antitumor activity of tetrathiomolybdate in patients with advanced kidney cancer and confirmed its efficacy in the treatment of kidney cancer in combination with other antiangiogenic therapies (Redman, B. G., Esper, P.; Pan, Q.; Dunn, R. L.; Hussain, H. K.; Chenever, T.; Brewer, G. J.; Merajver, S. D. Clin. l Cancer Res., 2003, 9, 1666-1672).

### SUMMARY OF TME INVENTION

It has been found for the first time in accordance with the present invention that a group of organometallic molybdenum (II) complexes exhibit cytostatic activity against cancer cells. The present invention provides a method for treating cancer affecting mammals by administering an effective amount of the molybdenum (II) complex and pharmaceutical compositions containing said complexes.

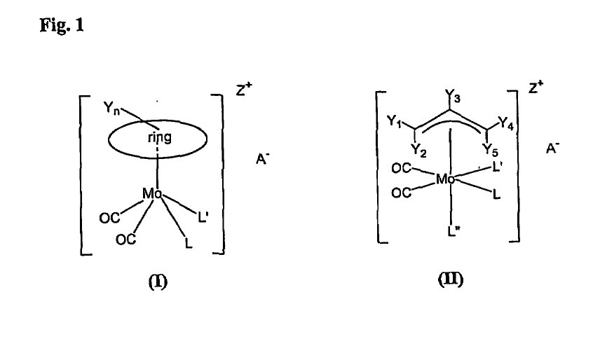

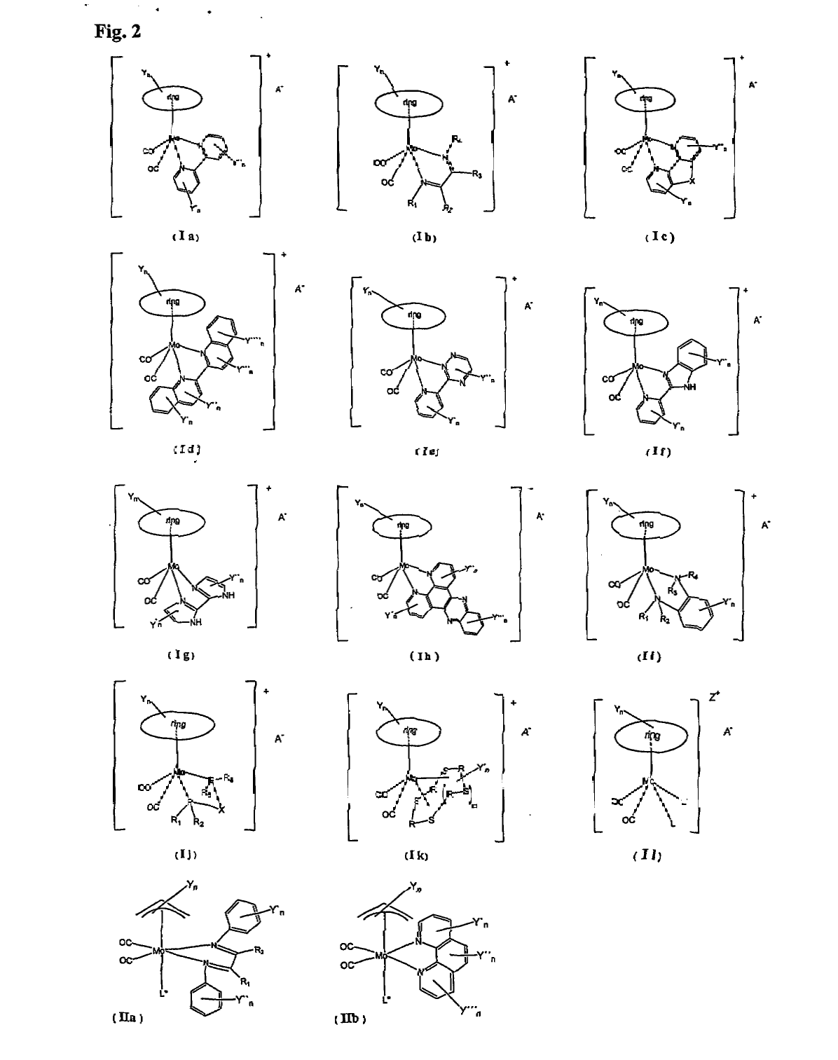

These compounds have the general formula (I) (FIG. 1) wherein,
“ring” represents either cyclopentadienyl or indenyl;
Yₙ represents n substituents which can be chosen, independently, from H, alkyl, alkenyl, alkoxy, aryl, halogen, haloallyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or one bidentate ligand with C, N, O, P or S donor atoms;
Z⁺ represents the overall charge of the Mo (II) complex, usually 1⁺ or 0;
A⁻ represents one suitable and pharmaceutically acceptable counter anion that equilibrate the complex charge when needed.

The invention also provides compounds of the formula (II) (FIG. 1), wherein,
Y₁, Y₂, Y₃, Y₄, Y₅ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or oneibidentate ligand with C, N, O, P or S donor atoms;
L″ represents one monodentate ligand coordinated via one C, N, O, P, S or halide donor atom;
Z⁺ represents the overall charge of the Mo (II) complex, usually 1⁺ or 0;
A⁻ represents one suitable and pharmaceutically acceptable counter anion that equilibrate the complex charge when needed.

### DETAILED DESCRIPTION

Molybdenum is an extremely versatile element, forming compounds in a wide range of readily interconvertible oxidation states. In biological systems molybdenum is an essential constituent of enzymes that catalyse redox reactions, like the oxidation of xanthine or sulfite (Kisker, C.; Schindelin, H.; Rees, D. C. Annu. Rev. Biochem. 1997, 66, 233-267) and the reduction of nitrate to molecular nitrogen (Sellmann, D. Angew. Chem. 1993, 32, 64-67). The biochemical importance of molybdenum is due to its ability to provide facile electron-transfer pathways and to form bonds with nitrogen-, oxygen- and sulfur-donors, thus interacting with various biomolecules. In its general chemistry molybdenum is very different from the common toxic heavy metals. such as cadmium, lead, and mercury. Molybdenum is ingested, transported, and excreted as an anion [MoO₄]₂⁻ which is structurally similar to phosphate and sulfate. Thus molybdenum, while having an essential biochemical role in various redox processes, does not combine sufficiently strongly with physiologically important compounds to have a serious blocking effect on metabolic processes and so its toxicity, certainly with regard to human beings, is low (Vyskocil, A.; Viau, C. J. Appl. Toxicology, 1999, 19, 185-192).

The following definitions are used unless otherwise described: Halide or halogen is understood as meaning fluoride, chloride, bromide or iodide; Alkyl, alkoxy, etc. denote both straight-chain or branched alkyl radicals; Alkenyl is understood as meaning unsaturated radical; Aryl is understood as meaning aromatic and fused aromatic radicals.

Specific values listed below for radicals, substituents and ligands are for illustration only; they do not exclude other defined values.

As used herein the following definitions define the stated terms:

“Organometallic compound” is an organic compound comprised of a metal attached directly to carbon (R-M).

“Coordination compound” is a compound formed by the union of a central metal atom or ion with ions or molecules called ligands or complexing agents.

“Ligand” or a “complexing agent” is a molecule, ion or atom that is attached to the central atom or ion of a coordination compound.

“Monodentate ligand” is a ligand having a single donor atom coordinated to the central metal atom or ion.

“Bidentate ligand” is a ligand having two donor atoms coordinated to the same central metal atom or ion.

“Molybdenum (II) complex” is a coordination compound including molybdenum as the central metal atom or ion, and the molybdenum has an oxidation state (II).

The present invention discloses organometallic molybdenum (II) complexes and the finding that such complexes have potent and selective antitumor activity.

Compounds disclosed by the invention include molybdenum (II) organometallic complexes having antitumor activity. Specifically the molybdenum (II) complex is a compound of the general formula (I), FIG. 1, wherein,
“ring” represents either cyclopentadienyl or indenyl;
Yₙ represents n substituents which can be chosen, independently, from H, allyl, alkenyl, alkoxy, aryl, halogen, haloalkyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or one bidentate ligand with C, N, O, P or S donor atoms;
Z⁺ represents the overall charge of the Mo (II) complex, usually 1⁺ or 0;
A⁻ represents one suitable and pharmaceutically acceptable counter anion that equilibrates the complex charge when needed.

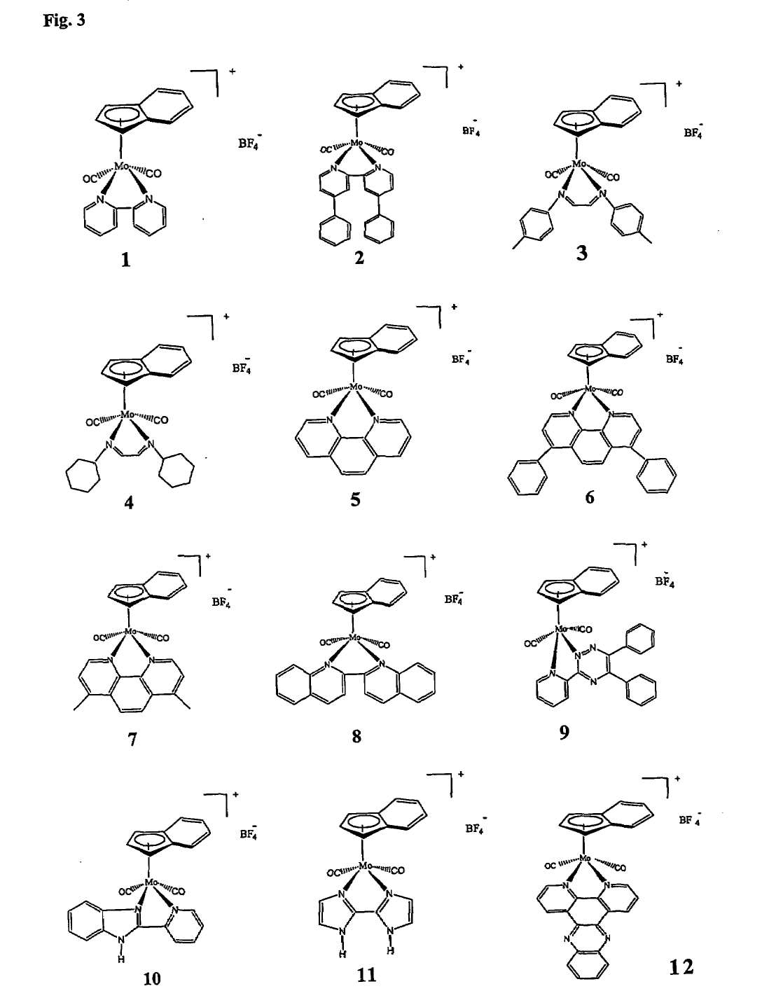

Specifically, the molybdenum (II) complex is a compound of formula Ia, FIG. 2, wherein, “ring” represents either cyclopentadienyl or indenyl;
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ia) can be [(η⁵-Ind)Mo(CO)₂bpy]BF₄ (compound 1) and [(η⁵-Ind)Mo(CO)₂(4,4′-Ph₂-2,2′-bpy)]BF₄ (compound 2);

Specifically, the molybdenum (II) complex is a compound of formula Ib, FIG. 2, wherein,
“ring” represents either cyclopentadienyl or indenyl;
Yₙ represents n substituents which can be chosen, independently, from H, alkyl, alkenyl, alkoxy, aryl, halogen, haloalkyl, amino, organosilane (SiR₃), CO₂R, C(O)R, CHRCO₂R′, CHRCO₂R′,
R₁, R₂, R₃, R ₄ represent substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ib) is [(η⁵-Ind)Mo(CO)₂(p-tolilDAB)]BF₄ (compound 3) and [(η⁵-Ind)Mo(CO)₂CYDAB]BF₄ (compound 4);

Specifically, the molybdenum (II) complex is a compound of formula Ic, FIG. 2, wherein,
“ring” represents either cyclopentadienyl or indenyl;
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
X represents O, CH₂, CH₂—CH₂, and CH═CH;
A⁻ represents one suitable and pharmaceutically acceptable counter anion that equilibrates the complex charge.

Specifically the compound of formula (Ic) is [(η⁵-Ind)Mo(CO)₂(1,10-phen)]BF₄ (compound 5), [(η⁵-Ind)Mo(CO)₂(4,7-Ph₂-1,10-phen)]BF₄ (compound 6) and [(η⁵-Ind)Mo(CO)₂(4,7-Me₂-1,10-phen)′]BF₄(compound 7);

Specifically, the molybdenum (II) complex is a compound of formula Id, FIG. 2, wherein,
“ring” represents either cyclopentadienyl or indenyl;
Yₙ, Y′ₙ, Y″ₙ, Y′″ₙ, Y″″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Id) is [(η⁵-Ind)Mo(CO)₂(2,2′-biq)]BF₄ (compound 8);

Specifically, the molybdenum (II) complex is a compound of formula Ie, FIG. 2, wherein,
“ring” represents either cyclopentadienyl or indenyl;
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ie) is [(η⁵-Ind)Mo(CO)₂ {5,6-Ph₂-3-(2-py)-1,2,4-Tz}]BF₄ (compound 9) and [(η⁵-Cp)Mo(CO)₂{5,6-Ph₂-3-(2-py)-1,2,4-Tz}]BF₄ (Compound 22).

Specifically, the molybdenum (II) complex is a compound of formula If, wherein,
“ring” represents either cyclopentadienyl or indenyl;
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (If) is [(η⁵-Ind)Mo(CO)₂{(2-py)-benz}]BF₄ (compound 10);

Specifically, the molybdenum (II) complex is a compound of formula Ig, FIG. 2, wherein,
“ring” represents either cyclopentadienyl or indenyl;
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ig) is [(η⁵-Ind)Mo(CO)₂(2,2′-H₂biim)]BF₄ (compound 11);

Specifically, the molybdenum (II) complex is a compound of formula Ih, FIG. 2, wherein,
“ring” represents either cyclopentadienyl or indenyl;
Yₙ, Y′ₙ, Y″ₙ, Y′″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ih) is: [(η⁵-Ind)Mo(CO)₂dppz]BF₄ (compound 12);

Specifically, the molybdenum (II) complex is a compound of formula Ii, FIG. 2, wherein,
“ring” represents either cyclopentadienyl or indenyl;
Yₙ, Y′ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
R₁, R₂, R₃, R₄, represent substituents which can be chosen, independently, from H, alkyl, aryl, organosilane (SiR₃), CO₂R, C(O)R, CHRCO₂R′, CHROH, cyano or nitro;
A⁻ represents one suitable and pharmaceutically acceptable counter anion that equilibrates the complex charge.

Specifically the compound of formula (Ii) is: [(η⁵-Ind)Mo(CO)₂{1,2-Ph(NH₂)₂}]BF₄ (compound 13);

Specifically, the molybdenum (II) complex is a compound of formula Ij, FIG. 2, wherein,
“ring” represents either cyclopentadienyl or indenyl;
Yₙ represents n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
R₁, R₂, R₃, R₄ represent substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
X represents O, CH₂, CH₂—CH₂, and CH═CH;
A⁻ represents one suitable and pharmaceutically acceptable counter anion that equilibrates the complex charge.

Specifically the compound of formula (Ij) is [(η⁵-Ind)Mo(CO)₂dppe]BF₄ (compound 14) and [(η⁵-Cp)Mo(CO)₂dppe] BF₄ (Compound 21);

Specifically, the molybdenum (II) complex is a compound of formula Ik, FIG. 2, wherein,
“ring” represents either cyclopentadienyl or indenyl;
Yₙ, Y′ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
R represents an alkyl or alkenyl chain;
m=0 or integer number;

0A⁻ represents one suitable and pharmaceutically acceptable counter anion that equilibrates the complex charge.

Specifically the compound of formula (Ik) is [(η⁵-Ind)Mo(CO)₂trithiane]BF₄ (compound 15); [(η³-Ind)Mo(CO)₂tten]BF₄ (compound 16), [(η⁵-Ind)Mo(CO)₂(1,4,7,10-tetrt)]BF₄ (compound 17), [(η⁵-Cp)Mo(CO)₂trithiane]BF₄ (Compound 19) and [(η⁵-Cp)Mo(CO)₂tten]BF₄ (Compound 20);

Specifically, the molybdenum (II) complex is a compound of formula Il, FIG. 2, wherein,
“ring” represents either cyclopentadienyl or indenyl;
Yₙ represents n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L and L′ represent two independent monodentate ligands coordinated via C, N, O, P, S, or halide donor atoms;
Z⁺ represents the overall charge of the Mo (II) complex, usually 1⁺ or 0;
A⁻ represents one suitable and pharmaceutically acceptable counter anion that equilibrates the complex charge when needed.

Specifically the compound of formula (Il) is [(η⁵-Ind)Mo(CO)₂(NCMe)₂]BF₄ (compound 18);

Specifically, the molybdenum (II) complex is a compound with the general formula (II), FIG. 1, wherein,
Y₁, Y₂, Y₃, Y₄, Y₅ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or one bidentate ligand with C, N, O, P or S donor atoms;
L″ represents one monodentate ligand coordinated via one C, N, O, P, S or halide donor atom; Z⁺ represents the overall charge of the Mo (II) complex, usually 1⁺ or 0;
A⁻ represents one suitable and pharmaceutically acceptable counter anion that equilibrates the complex charge when needed.

Specifically, the molybdenum (II) complex is a compound of formula IIa, FIG. 2, wherein,
Yₙ, Y′ₙ, Y″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
R₁, R₂ represent substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro; L″ represents one monodentate ligand coordinated via one C, N, O, P, S or halide donor atom;

Specifically the compound of formula (IIa) is (η³-C₃H₅)Mo(CO)₂(dimethyl-p-tolilDAB)Br (Compound 23).

Specifically, the molybdenum (II) complex is a compound of formula IIb, FIG. 2, wherein, Yₙ, Y′ₙ, Y″ₙ, Y′″ₙ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L″ represents one monodentate ligand coordinated via one C, N, O, P, S or halide donor atom;

Specifically the compound of formula (IIb) is (η³-C₃H₅)Mo(CO)₂(1,10-phen)Br (Compound 24) and (η³-C₃H₅)Mo(CO)₂(4,7-diphenyl-1,10-phen)Br (Compound 25).

The medicinal agent of the invention can be formulated as a pharmaceutical composition and be administered to an animal host such as a human patient, in a variety of forms adapted to the chosen route of administration, i.e., orally, rectally or parenterally, e.g., intravenously (i.v.), subcutaneously, intramuscularly, intrapleurally, intraperitoneally, intrafocally or perifocally.

The pharmaceutical compositions normally consist of the active agents of this invention and non-toxic, pharmaceutically acceptable vehicles used as an admixture in solid, semisolid, or liquid form, or as an encasing composition, for example, in the form of a capsule, a tablet coating, a bag, or some other container for the active agent. In this connection, the vehicle can serve, for example, as an intermediary for the medicine absorption by the body, as an auxiliary formulating agent, sweetener, flavor-ameliorating agent, coloring agent or preservative.

Suitable for oral administration are for example, tablets, dragees, hard and soft gelatin capsules, dispersible powders, granules, aqueous and oil suspensions, emulsions, solutions, and syrups.

Tablets can contain inert diluents such as calcium carbonate, calcium phosphate, sodium phosphate or lactose; granulating and distributing agents, such as corn starch or alginates; binders such as amylose, gelatin, or acacia gum and lubrificants, such as aluminum stearate, or magnesium stearate, talc or silicone oil. Optionally, the tablets are provided with a coating which can also have such a character that effects a delayed dissolution and reabsorption of the medicinal agent in the gastrointestinal tract and thus, for example, provides improved compatibility or a longer duration of effectiveness.

Gelatin capsules can contain the active agent in a mixture with a solid diluent (e.g. calcium carbonate or kaolin) or an oily diluent (e.g. olive, peanut, or paraffin oil).

Suitable suspensions agents are for instance, sodium carboxymethylcellulose, methylcellulose, hydroxypropylcellulose, sodium alginate, polyvinylpyrrolidone, tragacanth gum or acacia gum;

Suitable dispersing and wetting agents are for example polyoxyethylene stereate, heptadecaethyleneoxycetanol, polyoxyethylene, sorbitol monooleate, polyoxyethylen sorbitan monooleate or lechitin;

Suitable preservatives are for example, methyl or propyl hydroxybenzoate;

Suitable flavoring agents or sweeteners are for instance, sucrose, lactose, dextrose or invert sugar syrup.

Oily suspensions can contain, for example, peanut, olive, sesame, coconut, or paraffin oil, as well as thickeners, such as beeswax, hard paraffin or cetyl alcohol, sweeteners, flavoring agents and/or anti-oxidants.

Water dispersible powders and granules contain the active agent in a mixture with dispersing, wetting, and suspension agents, e.g., the aforementioned materials and/or dimethyl sulfoxide, as well as in a mixture with sweeteners, flavoring agents and/or coloring agents.

Emulsions can contain for example, olive, peanut, or paraffin oil in addition to emulsifiers, such as acacia gum, tragacanth gum, phosphatides, sorbitan monooleate or polyoxyethylene sorbitan monooleate, sweeteners and/or flavoring agents.

Suitable for rectal applications are suppositories produced with the aid of binders melting at rectal temperature, for example, cocoa butter or polyethylene glycols.

The medicinal agents can be used parenterally as sterile isotonic sodium chloride solutions or other solutions. To attain uniform dissolution or suspension, a solubilizer is preferably added, such as dimethyl sulfoxide.

In all forms of administrations the medicinal agents of this invention can furthermore contain buffer substances e.g., sodium bicarbonate or tris(hydroxymethyl) aminomethane.

In addition to the molybdenum (II) complexes employed in this invention, the medicinal agents can contain one or more other pharmacologically active components of other cytostatically effective groups of medicines e.g. alkylating agents or anti-metabolites as well as cytostatic alkaloids, antibiotics, enzymes and heavy metal compounds. Furthermore the medicinal agents can optionally contain substances having an imunopressive effect and vitamins. The above mentioned additives can also be added in separate pharmaceutical preparations or in the form of combination preparations to the active agents of the present invention.

Useful dosages of the compounds of the present invention can be determined by comparing their in vitro activity and in vivo activity in animal models. Methods for extrapolation of effective dosages in mice and other animals, to humans are known to the art (U.S. Pat. No. 4,938,949 or Guidance Document on using in vitro data to estimate in vivo starting doses for acute toxicity, National Institute of Environmental Health Sciences, U.S. Public Health Service).

The amount of the composition required for use in treatment will vary not only with the particular compound selected but also with the route of administration, the nature of the condition being treated and the age and condition of the patient and will be ultimately at the discretion of the attendant physician or clinician.

The active agent content in the pharmaceutical compositions of the invention is ordinarily 0.01%-95% by weight, preferably 0.1-85% by weight based on the finished medicine, i.e. the final pharmaceutical formulation. The desired dose may conveniently be presented in a single dose or as divided doses, administered at appropriate intervals, for example, as two, three, four or more sub-doses per day. The sub-dose itself may be further divided into a number of discrete loosely spaced administrations. If present in unit dosage form, the medicinal agents of the invention contain 1 mg to 10.000 mg, preferably 5 mg to 7.500 mg of active agent.

The antitumor activity of the compositions of the invention can be determined using assays that are know in the art, or can be determined using assays similar to those described in the following examples.

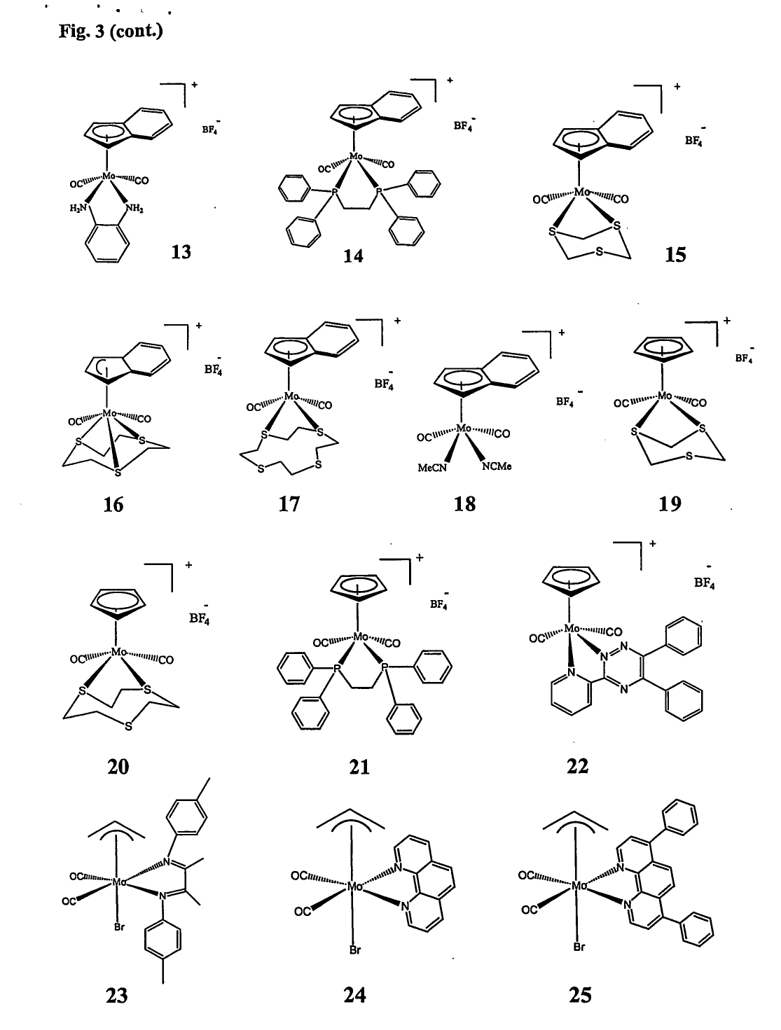

The present invention is further illustrated by the examples depicted in FIG. 3 which are illustrative only, and were prepared in accordance with the procedures that are given below. With few exceptions stated where appropriate, said examples are unknown in prior art of chemical synthesis and none of them has been previously used for the purposes that are disclosed in the present invention.

### EXAMPLES

### Abbreviations

Cp: η⁵-cyclopentadienyl; Ind: η⁵-indenyl; bpy: 2,2′-bipyridine; Ph: phenyl; Me: methyl; DAB: diazabutadiene; CYDAB: 1,4-bis(cyclohexyl)diazabutadiene; phen: 1,10-phenanthroline; Py: pyridine; Tz: Triazine; Benz: benzimidazol; Biq: biquinoline; 2,2′-H₂biim: 2,2′-bis-imidazol; dppz: dipyrido[3,2-a:2′3′-c]phenazine; Ph(NH₂)₂: 1,2-diaminobenzene; dppe: 1,2-bis(diphenilphosphino)ethane; trithiane: trithiocyclohexane; tten: trithiocyclononane; tetrt: tetrathiocyclododecane; dme: 1,2-dimethoxyethane; MeCN: acetonitrile;

### Materials and Methods

All experiments were carried under nitrogen atmosphere using standard Schlenk techniques. Solvents were dried by standard procedures, distilled and kept under nitrogen and molecular sieves. Diethyl ether, 1,2-dimethoxiethane and hexane were dried over sodium wire and benzophenone ketyl, refluxed and distilled. Dichloromethane and acetonitrile were distilled over CaH₂.

Infrared spectra were recorded on a Unicam Mattson Mod 7000 FTIR spectrophotometer using KBr pellets or in solution. The band intensities were represented as weak (w), medium (m), strong (s) and very strong (vs);

¹H NMR and ¹³C NMR spectra were measured on a Brüker AMX 300 and 75 MHz, respectively; Microanalyses were performed by Eng. Conceição Almeida at the Elemental Analysis Service of ITQB (Instituto de Tecnologia Química e Biológica) on a Carlo Erba Mod 1106.

(η⁵-Ind)Mo(CO)₂(η³-C₃H₅) and (η⁵-Cp)Mo(CO)₂(η³-C₃H₅) were used as starting materials and were prepared according to the literature (Ascenso, J. A.; De Azevedo, C. G.; Gonçalves, I. S.; Herdtweck, E.; Moreno, D.; Romão, C. C.; Zühlke, J. Organometallics, 1994, 13, 429-431);

The indenyl and cyclopentadienyl monocations of general formula [IndMo(CO)₂L₂]⁺ were prepared using a well established reaction sequence (Ascenso, J. A.; Gonçalves, I. S.; Herdtweck, E.; Romão, C. C. J. Organomet. Chem. 1996, 508, 169-181);

The allyl complexes were prepared by substitution of the MeCN ligands in (η³-C₃H₅)MoBr(CO)₂(NCMe)₂ with the appropriate ligands (L), a process well established in the literature. The ligands were obtained from Aldrich or prepared according to literature procedures.

The structural formulae of some specific compounds under the following examples (examples 1-7) are given in FIG. 3.

### Example 1

### Indenyl Molybdenum (II) Complexes with Nitrogen Ligands

[(η⁵-Ind)Mo(CO)₂bpy]BF₄ (Compound 1)

A solution of (η⁵-Ind)Mo(CO)₂(η³-C₃H₅) (0.50 g, 1.6 mmol) in CH₂Cl₂ was treated with HBF₄.Et₂O (1 eq.). After 10 minutes dimethoxyethane (dme) was added in excess and the reaction was left for 15 minutes. 0.31 g (2 mmol) of 2,2′-bpy were added and the reaction was left for 2 hours at room temperature. After concentration to about 5 ml and addition of Et₂O, a red complex precipitated. The mixture was filtered and the residue recrystallized from CH₂Cl₂/Et₂O (η=98%). This method is a slight modification of the published procedure (Ascenso, J. R.; Gonçalves, I. S.; Herdtweck, E.; Romão, C. C. J. Organomet. Chem. 1996, 508, 169-181) and the analytical data matched that of the original compound. A drawing of the structure and physical data are given in Table 1.

[(η⁵-Ind)Mo(CO)₂(4,4′-Ph₂-2,2′-bpy)]BF₄ (compound 2);

A solution of (η⁵-Ind)Mo(CO)₂(η³-C₃H₅) (0.2 g, 0.65 mmol) in CH₂Cl₂ was treated with HBF₄.Et₂O (1 eq.). After 10 minutes dme was added in excess and the reaction was left for 15 minutes. 0.25 g (0.8 mmol) of 4,4′-diphenyl-2,2′-bpy were added and the reaction was left for 2 hours at room temperature. After concentration to about 5 ml and addition of Et₂O, a ruby complex precipitated. The mixture was filtered and the residue recrystallized from CH₂Cl₂/Et₂O (η=90%); A drawing of the structure and physical data are given in Table 1.

[(η⁵-Ind)Mo(CO)₂(p-tolilDAB)]BF₄ (Compound 3)

A solution of (η⁵-Ind)Mo(CO)₂(η³-C₃H₅) (0.50 g, 1.6 mmol) in CH₂Cl₂ was treated with HBF₄.Et₂O (1 eq.). After 10 minutes dme was added in excess and the reaction was left for 15 minutes. 0.47 g (2 mmol) of p-tolilDAB were added and the reaction was left for 2 hours at room temperature. After concentration to about 5 ml and addition of hexane, a dark purple complex precipitated. The mixture was filtered and the residue recrystallized from CH₂Cl₂/hexane (η=90%). A drawing of the structure and physical data are given in Table 1.

### [(η⁵-Ind)Mo(CO)₂(CYDAB)]BF₄ (Compound 4)

A solution of (η⁵-Ind)Mo(CO)₂(η³-C₃H₅) (0.50 g, 1.6 mmol) in CH₂Cl₂ was treated with HBF₄.Et₂O (1 eq.). After 10 minutes dme was added in excess and the reaction was left for 15 minutes. 0.47 g (2 mmol) of cyclohexyldiazabutadiene were added and the reaction was left for 2 hours at room temperature. After concentration to about 5 ml and addition of hexane, a dark-purple complex precipitated. The mixture was filtered and the residue recrystallized from CH₂Cl₂/hexane (η=90%).

A drawing of the structure and physical data are given in Table 1.

[(η⁵-Ind)Mo(CO)₂phen]BF₄ (Compound 5)

A solution of (η⁵-Ind)Mo(CO)₂(η³-C₃H₅) (0.20 g, 0.65 mmol) in CH₂Cl₂ was treated with HBF₄.Et₂O (1 eq.). After 10 minutes dme was added in excess and the reaction was left for 15 minutes. 0.14 g (0.8 mmol) of 1,10-phenantroline were added and the reaction was left for 2 hours at room temperature. After concentration to about 5 ml and addition of Et₂O, a ruby complex precipitated. The mixture was filtered and the residue recrystallized from CH₂Cl₂/Et₂O (η=90%). A drawing of the structure and physical data are given in Table 1.

[(η⁵-Ind)Mo(CO)₂(4,7-Ph₂-1,10-phen)]BF₄ (Compound 6)

A solution of (η⁵-Ind)Mo(CO)₂(η³-C₃H₅) (0.20 g, 0.65 mmol) in CH₂Cl₂ was treated with HBF₄.Et₂O (1 eq.). After 10 minutes dme was added in excess and the reaction was left for 15 minutes. 0.27 g (0.8 mmol) of 4,7-diphenil-1,10-phenantroline were added and the reaction was left for 2 hours at room temperature. After concentration to about 5 ml and addition of Et₂O, a ruby complex precipitated. The mixture was filtered and the residue recrystallized from CH₂Cl₂/Et₂O (η=90%). A drawing of the structure and physical data are given in Table 1.

[(η⁵-Ind)Mo(CO)₂(4,7-Me₂-1,10-phen)]BF₄ (Compound 7)

A solution of (η⁵-Ind)Mo(CO)₂(η³-C₃H₅) (0.20 g, 0.65 mmol) in CH₂Cl₂ was treated with HBF₄.Et₂O (1 eq.). After 10 minutes dme was added in excess and the reaction was left for 15 minutes. 0.17 g (0.8 mmol) of 4,7-dimethyl-1,10-phenantroline were added and the reaction was left for 2 hours at room temperature. After concentration to about 5 ml and addition of Et₂O, a red complex precipitated. The mixture was filtered and the residue recrystallized from CH₂Cl₂/Et₂O (η=90%). A drawing of the structure and physical data are given in Table 1.

[(η⁵-Ind)Mo(CO)₂(2,2′-biq)]BF₄ (Compound 8)

A solution of (η⁵-Ind)Mo(CO)₂(η³-C₃H₅) (0.27 g, 0.87 mmol) in CH₂Cl₂ was treated with HBF₄.Et₂O (1 eq.). After 10 minutes ddme was added in excess and the reaction was left for 15 minutes. 0.33 g (0.84 mmol) of 2,2′-biquinoline were added and the reaction was left for 2 hours at room temperature. After concentration to about 5 ml and addition of Et₂O, a deep blue complex precipitated. The mixture was filtered and the residue recrystallized from CH₂Cl₂/Et₂O (η=90%). A drawing of the structure and physical data are given in Table 1.

[(η⁵-Ind)Mo(CO)₂{5,6-Ph₂-3-(2-py)-1,2,4-Tz}]BF₄ (Compound 9)

A solution of (η⁵-Ind)Mo(CO)₂(η³-C₃H₅) (0.50 g, 1.6 mmol) in CH₂Cl₂ was treated with HBF₄.Et₂O (1 eq.). After 10 minutes dme was added in excess and the reaction was left for 15 minutes. 0.62 g (2 mmol) of 5,6-diphenyl-3-(2-pyridil)-1,2,4-triazine were added and the reaction was left for 2 hours at room temperature. After concentration to about 5 ml and addition of hexane, a purple complex precipitated. The mixture was filtered and the residue recrystallized from CH₂Cl₂/hexane (η=90%). A drawing of the structure and physical data are given in Table 1.

[(η⁵-Ind)Mo(CO)₂{2-(2-py)-benz}]BF₄ (Compound 10)

A solution of (η⁵-Ind)Mo(CO)₂(η³-C₃H₅) (0.20 g, 0.65 mmol) in CH₂Cl₂ was treated with HBF₄.Et₂O (1 eq.). After 10 minutes dme was added in excess and the reaction was left for 15 minutes. 0.16 g (0.8 mmol) of 2-(2-pyridil)-benzimidazol were added and the reaction was left for 2 hours at room temperature. After concentration to about 5 ml and addition of hexane, a red complex precipitated. The mixture was filtered and the residue recrystallized from CH₂Cl₂/hexane (η=90%). A drawing of the structure and physical data are given in Table 1.

[(η⁵-Ind)Mo(CO)₂(2,2′-H₂biim)]BF₄ (Compound 11)

A solution of (η⁵-Ind)Mo(CO)₂(η³-C₃H₅) (0.25 g, 0.81 mmol) in CH₂Cl₂ was treated with HBF₄.Et₂O (1 eq.). After 10 minutes dme was added in excess and the reaction was left for 15 minutes. 0.13 g (1 mmol) of 2,2′-bis-imidazol were added and the reaction was left for 2 hours at room temperature. After concentration to about 5 ml and addition of Et₂O, an orange complex precipitated. The mixture was filtered and the residue recrystallized from CH₂Cl₂/Et₂O (η=90%). A drawing of the structure and physical data are given in Table 1.

[(η⁵-Ind)Mo(CO)₂dppz]BF₄ (Compound 12)

A solution of (η⁵-Ind)Mo(CO)₂(η³-C₃H₅) (0.11 g, 0.35 mmol) in CH₂Cl₂ was treated with HBF₄.Et₂O (1 eq.). After 10 minutes dme was added in excess and the reaction was left for 15 minutes. 0.09 g (0.31 mmol) of dppz were added and the reaction was left for 2 hours at room temperature. After concentration to about 5 ml and addition of Et₂O, a ruby complex precipitated. The mixture was filtered and the residue recrystallized from CH₂Cl₂/Et₂O, (η=75%). A drawing of the structure and physical data are given in Table 1.

### [(η⁵-Ind)Mo(CO)₂{1,2-Ph(NH₂)₂}]BF₄ (Compound 13)

A solution of (η⁵-Ind)Mo(CO)₂(η³-C₃H₅) (0.23 g, 0.74 mmol) in CH₂Cl₂ was treated with HBF₄.Et₂O (1 eq.). After 10 minutes dme was added in excess and the reaction was left for 15 minutes. 0.92 g (0.85 mmol) of 1,2-diaminobenzene were added and the reaction was left for 2 hours at room temperature. A partially insoluble orange solid precipitated and full precipitation of complex was obtained after addition of Et₂O (η=90%); A drawing of the structure and physical data are given in Table 1.

### [(η⁵-Ind)Mo(CO)₂(NCMe)₂]BF₄ (Compound 18)

A solution of (Θ⁵-Ind)Mo(CO)₂(η³-C₃H₅) (0.25 g, 0.81 mmol) in CH₂Cl₂ was treated with HBF₄.Et₂O (1 eq.). After 10 minutes dme was added in excess and the reaction was left for 15 minutes. 5 ml of acetonitrile were added and the reaction was left for 2 hours at room temperature. After concentration to about 5 ml and addition of Et₂O, an orange complex precipitated. The mixture was filtered and the residue recrystallized from CH₂Cl₂/Et₂O (η=96%). This method is a slight modification of the published procedure (Green, M., Greenfield, S., Kersting, M., J. Chem. Soc. Chem. Commun., 1985, 18). The analytical data matched that of the original compound. A drawing of the structure and physical data are given in Table 1.

### Example 2

### Indenyl Molybdenum (II) Complexes with Phosphorus Ligands

[(η⁵-Ind)Mo(CO)₂dppe]BF₄ (Compound 14)

A solution of (η⁵-Ind)Mo(CO)₂(η³-C₃H₅) (0.50 g, 1.6 mmol) in CH₂Cl₂ was treated with HBF₄.Et₂O (1 eq.). After 10 minutes dme was added in excess and the reaction was left for 15 minutes. 0.79 g (2 mmol) of dppe were added and the reaction was left for 2 hours at room temperature. After concentration to about 5 ml and addition of diethyl ether, a yellow complex precipitated. The mixture was filtered and the residue recrystallized from CH₂Cl₂/Et₂O (η=98%). This method is a slight modification of the published procedure (Bottrill, M.; Green, M.; J. Chem. Soc. Dalton Trans. 1977, 2365). The analytical data matched that of the original compound. A drawing of the structure and physical data are given in Table 1.

### Example 3

### Indenyl Molybdenum (II) Complexes with Sulfur Ligands

[(η⁵-Ind)Mo(CO)₂trithiane]BF₄ (Compound 15)

A solution of (η⁵-Ind)Mo(CO)₂(η³-C₃H₅) (0.30 g, 0.97 mmol) in CH₂Cl₂ was treated with HBF₄.Et₂O (1 eq.). After 10 minutes dme was added in excess and the reaction was left for 15 minutes. 0.166 g (1.2 mmol) of trithiane were added and the reaction was left for 2 hours at room temperature. After concentration to about 5 ml and addition of diethyl ether, a red/orange complex precipitated. The mixture was filtered and the residue recrystallized from CH₂Cl₂/Et₂O (η=98%); A drawing of the structure and physical data are given in Table 1.

[(η⁵-Ind)Mo(CO)₂tten]BF₄ (Compound 16)

A solution of (η⁵-Ind)Mo(CO)₂(η³-C₃H₅) (0.06 g, 0.97 mmol) in CH₂Cl₂ was treated with HBF₄.Et₂O (1 eq.). After 10 minutes dme was added in excess and the reaction was left for 15 minutes. 0.045 g (0.25 mmol) of 1,4,7-trithiacyclononane (tten) were added and the reaction was left for 2 hours at room temperature. After concentration to about 5 ml and addition of diethyl ether, a green complex precipitated. The mixture was filtered and the residue recrystallized from CH₂Cl₂/Et₂O (η=98%). This method is a slight modification of the published procedure (Calhorda, M. J., Gamelas, C. A., Gonçalves, I. S., Herdtweck, E. Romão, C. C., Veiros, L. F., Organometallics, 1998, 17, 2597-2611). The analytical data matched that of the original compound. A drawing of the structure and physical data are given in Table 1.

[(η⁵-Ind)Mo(CO)₂(1,4,7,10-tetrt)]BF₄ (Compound 17)

A solution of (η⁵-Ind)Mo(CO)₂(η³-C₃H₅) (0.15 g, 0.48 mmol) in CH₂Cl₂ was treated with HBF₄.Et₂O (1 eq.). After 10 minutes dme was added in excess and the reaction was left for 15 minutes. 0.12 g (0.5 mmol) of 1,4,7,10-tetratiociclododecane (1,4,7,10-tetrt) were added and the reaction was left for 2 hours at room temperature. The partially insoluble orange complex was obtained after concentration and addition of diethyl ether. The residue was recrystallized from CH₂Cl₂/Et₂O (η=98%). A drawing of the structure and physical data are given in Table 1.

### Example 4

### Cyclopentadienyl Molybdenum (II) Complexes with Sulfur Ligands

[(η⁵-Cp)Mo(CO)₂trithiane]BF₄ (Compound 19)

A solution of (η⁵-Cp)Mo(CO)₂(η³-C₃H₅) (0.250 g, 0.97 mmol) in CH₂Cl₂ was treated with HBF₄.Et₂O (1 eq.). After 10 minutes dme was added in excess and the reaction was left for 15 minutes. 0.13 g (0.97 mmol) of 1,3,5-trithiane (tt) were added and the reaction was left for 2 hours at room temperature. After concentration to about 5 ml and addition of Et₂O, an orange complex precipitated. The mixture was filtered and the residue recrystallized from CH₂Cl₂/Et₂O (η=90%). A drawing of the structure and physical data are given in Table 1.

[(η⁵-Cp)Mo(CO)₂tten]BF₄ (Compound 20)

A solution of (η⁵-Cp)Mo(CO)₂(η³-C₃H₅) (0.347 g, 1.35 mmol) in CH₂Cl₂ was treated with HBF₄.Et₂O (1 eq.). After 10 minutes dme was added in excess and the reaction was left for 15 minutes. 0.24 g (1.35 mmol) of 1,4,7-trithiacyclononane (tten) were added and the reaction was left for 2 hours at room temperature. After concentration to about 5 ml and addition of Et₂O, an orange complex precipitated. The mixture was filtered and the residue recrystallized from CH₂Cl₂/Et₂O (η=90%). A drawing of the structure and physical data are given in Table 1.

### Example 5

### Cyclopentadienyl Molybdenum (II) Complexes with Phosphorus Ligands

[(η⁵-Cp)Mo(CO)₂dppe]BF₄ (Compound 21)

A solution of (η⁵-Cp)Mo(CO)₂(η³-C₃H₅) (0.200 g, 0.77 mmol) in CH₂Cl₂ was treated with HBF₄.Et₂O (1 eq.). After 10 minutes dme was added in excess and the reaction was left for 15 minutes. 0.35 g (0.88 mmol) of 1,2-bis(diphenylphosphino)ethane were added and the reaction was left for 2 hours at room temperature. After concentration to about 5 ml and addition of Et₂O, a yellow complex precipitated. The mixture was filtered and the residue recrystallized from CH₂Cl₂/Et₂O (η=90%). This method is a slight modification of the published procedure (J. R., Markham, J.; Menard, K.; Cutler, A. Inorg. Chem. 1985, 24, 1581-1487). The analytical data matched that of the original compound. A drawing of the structure and physical data are given in Table 1.

### Example 6

### Cyclopentadienyl Molybdenum (II) Complexes with Nitrogen Ligands

[(η⁵-Cp)Mo(CO)₂{5,6-Ph₂-3-(2-py)-1,2,4-Tz}]BF₄ (Compound 22)

A solution of (η⁵-Cp)Mo(CO)₂(η³-C₃H₅) (0.350 g, 1.35 mmol) in CH₂Cl₂ was treated with HBF₄.Et₂O (1 eq.). After 10 minutes dme was added in excess and the reaction was left for 15 minutes. 0.434 g (1.40 mmol) of 5,6-diphenyl-3-(2-pyridil)-1,2,4-triazine were added and the reaction was left for 2 hours at room temperature. After concentration to about 5 ml and addition of Et₂O, a dark purple complex precipitated. The mixture was filtered and the residue recrystallized from CH₂Cl₂/Et₂O (η=90%). A drawing of the structure and physical data are given in Table 1.

### Example 7

### Allyl Molybdenum (II) Complexes with Nitrogen Ligands

(η³-C₃H₅)Mo(CO)₂(2,3-Me₂-p-tolilDAB)Br (Compound 23)

To a strred solution of the allyl complex Mo(η³-C₃H₅)(CO)₂(NCCH₃)₂Br (0.355 g, 1 mmol) in ethanol (10 ml) and under a nitrogen atmosphere was added 2,3-Me₂-p-tolilDAB (0.266 g, 1 mmol). The suspension was stirred for three hours. The dark blue solution was concentrated and placed at 4° C. in order to form a precipitate which was then washed, recrystallized from CH₂Cl₂/hexane and dried under vacuum (η=87%). A drawing of the structure and physical data are given in Table 1.

(η³-C₃H₅)Mo(CO)₂(1,10-phen)Br (Compound 24)

The allyl complex Mo(η³-C₃H₅(CO)₂(NCCH₃)₂Br (0.355 g, 1 mmol) and the 1,10-phenanthroline (0.180 g, 1 mmol) were added to ethanol (10 ml) under a nitrogen atmosphere. The suspension was stirred for five hours. The red precipitate was separated from the solution by filtration. The precipitate was washed several times with small amounts of ether and dried under vacuum (η=90%). A drawing of the structure and physical data are given in Table 1.

(η³-C₃H₅)Mo(CO)₂(4,7-diphenyl-1,10-phen)Br (Compound 25)

The allyl complex Mo(η³-C₃H₅)(CO)₂(NCCH₃)₂Br (0.355 g, 1 mmol) and the 4,7-diphenyl-1,10-phenanthroline (0.332 g, 1 mmol) were added to ethanol (10 ml) under a nitrogen atmosphere. The suspension was stirred for five hours. The red precipitate was separated from the solution by filtration. The precipitate was washed several times with small amounts of ether and dried under vacuum (η=85%). A drawing of the structure and physical data are given in Table 1.

### In Vitro Cytotoxic Assays

In accordance to the present invention it has been determined that molybdenum (II) complexes exhibit cancerostatic activity as shown in the in vitro testing. The cytotoxic activity of theses complexes was evaluated against 6 different cell lines, using the MTT assay (3-(4,5-dimethylthiazol-2-yl)-2,5-diphenyltetrazolium bromide assay) (Mosmann, T. J. Immunol. Methods, 1983, 65, 55-63) to measure the cell viability.

### Cell Lines and Culture Conditions

Cell lines were routinely propagated in 75 cm² tissue culture flasks (SARSTEDT, Leicester, U.K.), in a humidified atmosphere of 5% CO₂ in air at 37° C., and were trypsinized and harvested into new medium every 2-4 days, just before confluence. Cell lines were cultured for a minimum of two passages after thawing prior to experimentation.

The Ehrlich ascites mouse tumor cell line was purchased at ECACC (European Collection of Cell culture) and propagated in NCTC-135 medium (Sigma, ref. N3262) 2 mM in L-glutamine, supplemented with 10% heat-inactivated fetal bovine serum (FBS) and 1% penicillin/streptomycin.

The tumoral MKN45 gastric and HT-29 colorectal human cell lines were purchased at ECACC. They were cultured in RPMI-1640 medium supplemented with glutamax (Gibco, ref 61870-044), 10% fetal bovine serum (FBS) and gentamicin (50 μg/ml).

The tumnoral GP-202 and GP-220 gastric human cell lines were established at IPATIMUP (Instituto de Patologia e Imunologia Molecular da Universidade do Porto). They were cultured in RPMI-1640 medium, supplemented with glutamax (Gibco, ref. 61870-044), 10% fetal bovine serum (FBS) and gentamicin (50 μg/ml).

The NMC-5 cells, secondary human lung fibroblasts, were purchased at ECACC. The cells were grown in MEM with Earls' Salt and L-glutamine (Gibco, ref. 61100-053), supplemented with 10% fetal bovine serum (FBS) and 1% neomycin.

### MIT Assay

This assay is based on the capacity of mitochondrial dehydrogenase enzymes in living cells to convert the yellow water soluble substrate (MTI) into a dark blue product which is quantified by spectrophotometric means. Briefly, exponentially growing cells were trypsinized, dispensed in sixplicates into 96-well tissue culture plates and allowed to attach overnight. The next day the cells were treated with various concentrations of the drug, ranging from 1 to 1000 μM. By addition of an adequate volume of a freshly prepared DMSO solution of the compound to the medium, the desired test concentrations were obtained. For. each test concentration and for the control which contained the corresponding amount of DMSO, 6 wells were used. After an incubation period of 3 hours the cells were carefully washed (twice) with phosphate buffer saline (PBS) and 100 μl of medium were added. The cells were incubated for 24 hours and 10 μl of a MTT solution (5 mg/ml) were added to each well. The tetrazolium/formazan reaction was allowed to proceed for 4 hours and the medium was carefully removed. The dark blue formazan crystals were dissolved by adding 150 μl of DMSO and agitating for 15 minutes in a plate shaker. The optical density was measured at 540 mn using a 96-well multiscanner autoreader. The percentage of survival was calculated using the formula: % survival=live cell number [test]/live cell number [control]×100. The IC₅₀ values were calculated by nonlinear regression analysis using the graphed Prism software (GraphPad Software, Inc., San Diego, Calif.) and are compiled in tables 2-4.

### RESULTS

### Synthesis and Characterization of Molybdenum (II) Complexes

The drawings of the structures and physical data (elemental analysis, infrared, mass spectrometry and ¹H-NMR spectral data) of compounds 1-25 are compiled in Table 1.

### Cytotbxic Effects of Molybdenum (II) Complexes

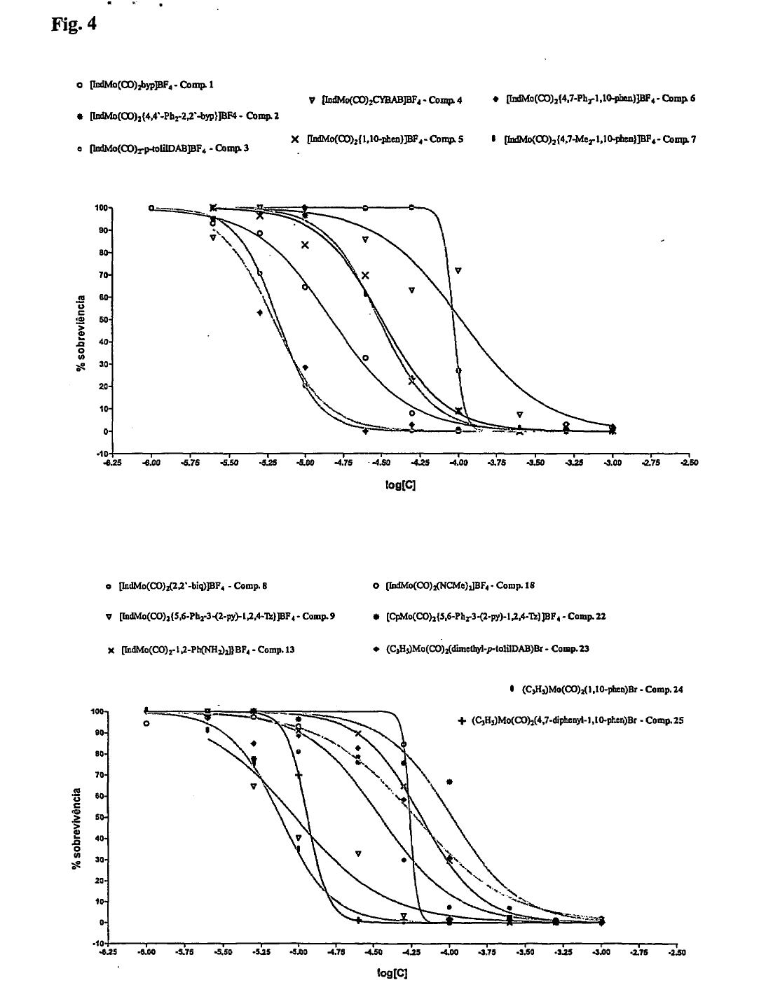

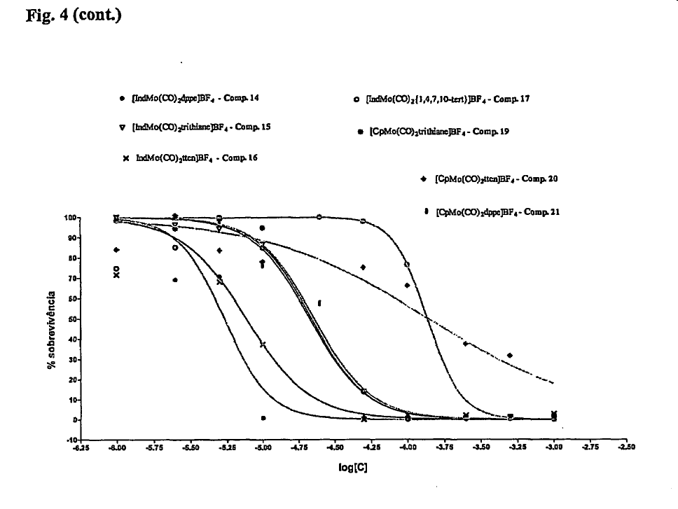

Using the colorimetric mitochondrial function-based MTT viability assay, we examined the effects of molybdenum (II) complexes against 6 different cell lines, by measuring the cellular proliferation at 8 different concentrations ranging from 1 to 1000 μM, 24 hours after removal of the drug. The IC₅₀ values were calculated from dose-response curves obtained by nonlinear regression analysis. FIG. 4 represent and compare dose-response curves obtained for several molybdenum compounds against the Ehrlich ascites mouse cell line.

As demonstrated by the IC₅₀ compiled in tables 2-4, the molybdenum (II) complexes are highly efficient cytotoxic agents against the in vitro growth of tumoral cells, specifically against the growth of the mouse Ehrlich ascites cancer cells (Table 2), the gastric and colon human cancer cells (Table 3) and the non-tumoral MRC-5 human fibroblast (Table 4).

In the case of the Ehrlich-ascites cell line, 22 molybdenum (II) complexes were tested (Table 2). All the indenyl molybdenum (II) complexes exhibit very good activities with IC₅₀ values ranging from 6 to 130 μM. The most potent effects were found for compounds 3, 6, 9, 14 and 16 with IC₅₀ values ranging from 6 to 10 μM. The common structural feature of these compounds is the presence of an aromatic ring at least 2 bonds apart from the metal. This observation suggests that intercalation might be a mechanism underlying the cytotoxic action of these compounds.

The equivalent cyclopentadienyl molybdenum (II) complexes (compounds 19 to 22) exhibit smaller activities when compared to the indenyl congeners suggesting that the indenyl ring contributes to the cancerostatic activity.

The IC₅₀ values obtained for complexes 6 and 8 against the colon and gastric human tumoral cell lines (Table 3) show that, at least for these complexes, the antiproliferative action is not cell specific. However, the human fibroblasts MRC-5 that were treated in the same manner with some of the molybdenum complexes, exhibited in general slightly higher IC₅₀ values (Table 4).

TABLE 1

| | | | | ¹H NMR data | |
|---|---|---|---|---|---|
| | | Elemental analysis | IR selected | (300 MHz, r.t., δ | Mass spectra |
| Compd. | Structure | Exp (Calc) | data (cm⁻¹) | ppm) | (m/z) |
| 1 | 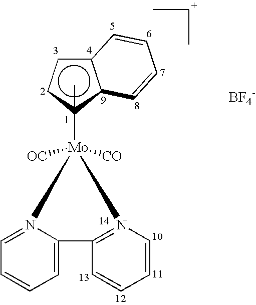 | C, 49.45; N, 5.46; H; 2.96(C, 49.38; N, 5.42; H, 3.05) | 3090 (w), 1970(vs) (CO),1892(vs) (CO),1062(vs) B—F | 9.36 (d, 2 H, H¹⁰), 8.30(d, 2 H, H¹³), 8.07 (t, 2 H,H¹¹), 7.57 (t, 2 H, H¹²),6.95 (m, 2 H, H⁵⁻⁸), 6.66(m, 2 H, H⁵⁻⁸), 6.47 (d,2 H, H^{1,3}), 5.47 (t, 1 H,H²), in NCMe-d₃ | |
| 2 | 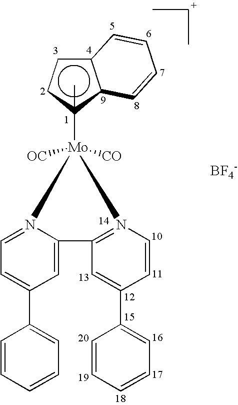 | C, 59.86; N, 4.37; H; 3.40(C, 59.85; N, 4.23; H, 3.50) | 1968 (vs, CO),1886 (vs, CO),1054 (vs,B—F) | 9.35 (d, 2 H, H¹⁰), 8.50(c, 4 H, H^{11,13}), 7.86 (c,6 H, H¹⁷⁻¹⁹), 7.62 (d, 4 H,H^{16,20}), 7.06, 6.80 (m,4 H, H⁵⁻⁸), 6.43 (d, 2 H,H^{1,3}), 5.43 (t, 1 H, H²), in(CH₂Cl₂-d₂) | |
| 3 | 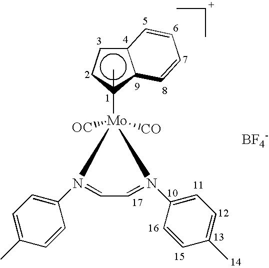 | C, 55.24; N, 4.46; H, 3.71(C, 54.94; N, 4.75; H, 3.93) | 2923 (w), 2019(vs, C═O),1976 (vs,C═O), 1513(m), 1459 (m),1445 (m), 1062(vs B—F) | 7.94 (s, 2 H, NCH), 7.38(d, 4 H, H¹²), 7.32 (m,2 H, H^{6,7}), 7.24 (d, 4 H,H¹¹), 7.12 (m, 2 H, H^{5,8}),6.09 (d, 2 H, H^{1,3}), 5.28(t, 1 H, H²), 2.48 (s, 6 H,CH₃) in CH₂Cl₂-d₂ | |
| 4 | 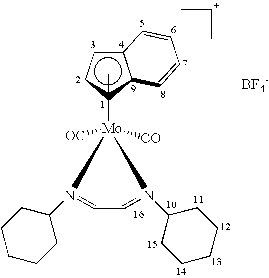 | | 2932 (s), 2856(s), 1994 (vs,CO), 1928 (vs,CO), 1083 (vs,B—F) | 7.95 (s, 2 H, H¹⁶), 7.50-7.39 (c, 4 H, H⁵⁻⁸), 6.06(d, 2 H, H^{1,3}), 5.52 (t, 1 H,H²), 2.30-1.00 (c, 10 H,H¹¹⁻¹⁵) in CH₂Cl₂-d₂ | 489.1 (M^{+.}[(IndMo(CO)₂(CYDAB)]⁺, 461.1(M^{+.} −28[(IndMoCO(CYDAB)]⁺ |
| 5 | 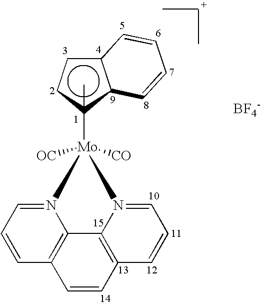 | C, 48.44; N, 4.10; H, 2.91(C, 51.72; N, 5.24; H, 2.83)Anal. Calc. (.1/2 CH₂Cl₂):C, 48.61; N, 4.82; H, 3.47 | 1970 (vs,C═O), 1875(vs, C═O),1430 (m), 1382(m), 1062 (vs,B—F), 844 (s),770 (m), 718(s) | 9.74 (d, 2 H, H¹⁰), 8.58(d, 2 H, H¹²), 8.02 (s, 2 H,H¹⁴), 7.99 (dd, 2 H, H¹¹),6.88, 6.33 (m, 4 H, H⁵⁻⁸),6.52 (d, 2 H, H^{1,3}), 5.44(t, 1 H, H²) in CH₂Cl₂-d₂ | ESI/MS (positivemode): 448.8 (M^{+.},[(IndMo(CO)₂(1,10-phen)]⁺) |
| 6 | 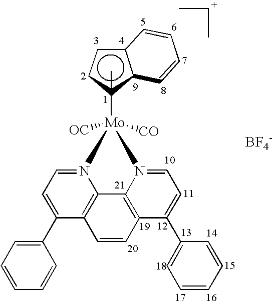 | C, 57.28; N, 3.62; H, 3.25(C, 59.85; N, 4.23; H, 3.50)Anal. Calc. (.1/2 CH₂Cl₂):C, 57.09; N, 3.97; H, 3.43 | 3100 (w), 1970(vs, C═O),1872 (vs,C═O), 1426(w), 1383 (w),1230 (w), 1062(vs, B—F), 763(m), 703 m) | 9.76 (d, 2 H, H¹⁰), 8.02(s, 2 H, H²⁰), 7.92 (d, 2 H,H¹¹), 7.60(c, 10 H, H¹⁴⁻¹⁸),7.05, 6.47 (m, 4 H,H⁵⁻⁸), 6.60 (d, 2 H, H^{1,3}),5.48 (t, 1 H, H²) inCH₂Cl₂-d₂ | 600.9 (M^{+.},[(IndMo(CO)₂(4,7-Ph₂-1,10-phen)]⁺),572.8, (M^{+.} −28,[(IndMo(CO)(4,7-Ph₂-1.10-phen)]⁺),544.9 (M^{+.} −56,[(IndMo(4,7-Ph₂-1.10-phen)]⁺). |
| 7 | 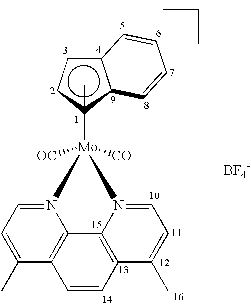 | C, 49.37; N, 4.74; H,3.73(C, 53.22; N, 4.97; H,3.38)Anal. Calc. (.1/4CH₂Cl₂): C, 52.0; N,4.80; H, 3.37 | 1970 (vs,C═O), 1873(vs, C═O),1423 (w),1383 (w),1058 (vs,B—F), 841 (w) | 9.44 (d, 2 H, H¹⁰), 8.06 (s,2 H, H¹⁴), 7.70 (d, 2 H,H¹¹), 6.81,6.28 (m, 4 H,H⁵⁻⁸), 6.39 (d, 2 H, H^{1,3}),5.36 (t, 1 H, H²), 2.86 (s,6 H, CH₃) in CH₂Cl₂-d₂ | |
| 8 | 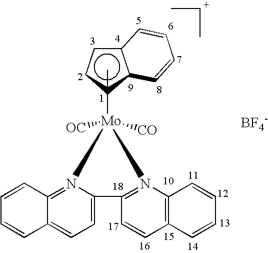 | C, 57.23; N, 4.55; H,3.15(C, 57.08; N, 4.59; H,3.14) | 1978 (vs,C═O), 1958(vs, C═O),1898 (vs,C═O), 1879(vs, C═O),1599 (s), 1510(s), 1063 (vs,B—F) | 9.29 (d, 2 H, H¹¹), 8.43 (d,2 H, H¹⁷), 7.78, 7.52 (c,8 H, H^{12-14,16}), 6.99, 6.71(m, 4 H, H⁵⁻⁸),6.37 (d,2 H, H^{1,3}), 5.34 (t, 1 H, H²)in CH₂Cl₂-d₂ | |
| 9 | 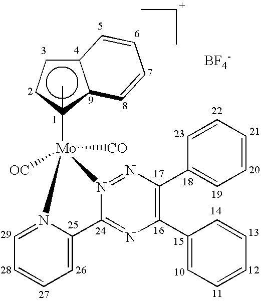 | C, 55.77 N, 8.68; H,3.22(C, 56.05; N, 8.43.; H,3.19) | 1982 (vs,C═O), 1899(vs, C═O),1372 (m),1057 (vs,B—F),772 (w),700 (w) | 9.45 (d, 1 H, H²⁹), 8.74 (d,1 H, H²⁶), 8.16 (dd, 1 H,H²⁷), 7.81 (dd, 1 H, H²⁸),7.71-7.42 (c, 14 H,H¹⁰⁻¹⁵⁺¹⁹⁻²³),7.08 (d, 1 H, H⁵),6.90 (d, 1 H, H⁸), 6.82 (c,2 H, H^{6,7}), 6.62 (m, 1 H,H³), 6.23 (m, 1 H, H¹),5.50 (dd, 1 H, H²) inCH₂Cl₂-d₂ | |
| 10 | 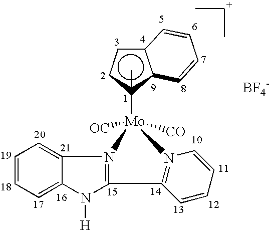 | | 1968 (vs, CO),1887 (vs, CO),1453 (vs),1083 (vs,B—F), 794 (m) | 11.10 (br, 1 H, NH), 9.18,8.97, 7.91, 6.72, 6.61 (m,8 H, H¹⁰⁻¹³⁺¹⁷⁻²⁰), 8.31,7.72 (d, 2 H, H⁵⁺⁸), 7.41,7.33 (dd, 2 H, H⁶⁺⁷), 6.46,6.33, 6.23 (m, 3 H, H¹⁻³)in CH₂Cl₂-d₂ | 464.0 (M^{+.},[IndMo(CO)₂{2-(2-py)-benz}]^{+.} |
| 11 | 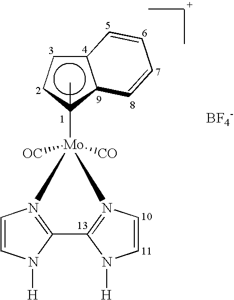 | C, 41.8; N, 11.4; H,2.60(C, 34.43; N, 8.70; H,2.10)Anal. Calc. (.2 CH₂Cl₂):(C, 34.67; N, 8.51; H,1.97) | 3130 (w),1963 (vs, CO),1880 (vs, CO),1322 (w),1084 (vs,B—F), 781 (m) | 11.6 (br, 2 H, NH), 7.52,7.22 (d, 4 H, H^{10,11}), 6.94,6.85 (m, 4 H,H⁵⁻⁸), 6.22(d, 2 H, H^{1,3}), 5.23 (t, 1 H,H²) in CH₂Cl₂-d₂ | 349.0 (M^{+.},[IndMo(CO)₂(2,2′-H₂biim)]^{+.} |
| 12 | 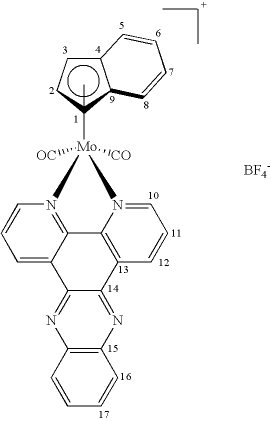 | C, 50.43; N, 7.75 H,3.28(C, 54.75; N, .8.81; H,2.69)Anal. Calc. (.CH₂Cl₂):C, 50.00; N, 7.77; H,2.63 | 1972 (vs,C═O), 1899(vs, C═O),1421 (w),1384 (w),1262 (w),1083(vs, B—F) | 9.79, 8.44, 8.10 (m, 10 H,H¹⁰⁻¹⁷), 7.00, 6.40 (m, 4 H,H⁵⁻⁸), 6.58 (d, 2 H, H^{1,3}),5.48 (t, 2 H, H²) inCH₂Cl₂-d₂ | |
| 13 | 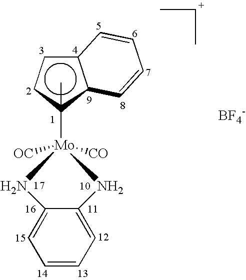 | | 1981 (vs, CO),1965 (vs, CO),1860 (vs, CO),1083 (vs,B—F) | 7.59, 6.81 (m, 4 H, H⁵⁻⁸),7.11, 7.02 (d, 4 H, H^{12,13}),6.28 (d, 2 H, H^{1,3}), 5.73(br, 4 H, NH₂), 5.19 (t,1 H, H₂). in acetone-d₆ | 375.0 (M^{+.},[IndMo(CO)₂{1,2-Ph(NH₂)₂}]^{+.} |
| 14 | 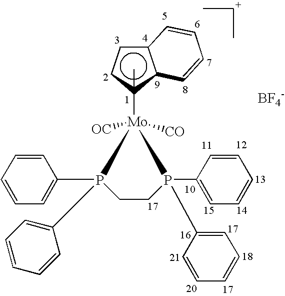 | C, 59.00; H, 4.00(C, 59.07; H, 4.15) | 1973 (vs, CO),1904 (vs, CO),1083 (vs,B—F) | 7.66-7.32 (m, 20 H,H¹¹⁻¹⁵),7.08 (m, 2 H, H⁵⁻⁸),6.01 (m, 2 H,H⁵⁻⁸), 5.59(d, 1 H,H^{1,3}), 5.28 (t, 1 H,H²), 2.64(br, 4 H, H¹⁷) inNCMe-d₃ | |
| 15 | 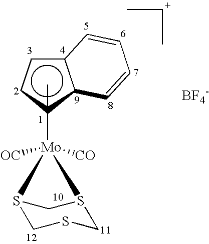 | C, 33.62; S, 19.45; H, 1.10(C, 34.17; S, 19.54;H, 2.66) | 3100 (w),1792 (vs, CO),1901 (vs, CO),1378 (w),1045 (vs,B—F), 835(w), 486 (w) | 7.84 (m, 2 H,H^{5,8}), 7.70(m, ⁴J_{H7H5} =3.1, 2 H, H^{6,7}),6.15 (d, 2 H,H^{1,3}), 5.22 (t,1 H, H²),4.83 (c, 2 H,H¹⁰),4.14 (br, 4 H,H^{11,12})in CH₂Cl₂-d₂ | |
| 16 | 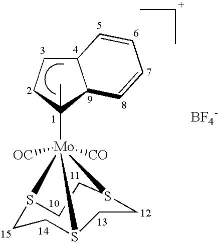 | C, 38.03; H, 4.10(C, 38.2; H, 3.58) | 1967 (vs, CO),1893 (vs, CO),1060 (vs,B—F) | 7.27 (t, 1 H, H²), 6.53-6.51 (m, 2 H, H⁵⁻⁸), 6.48-6.44 (m, 2 H, H⁵⁻⁸), 3.11-3.01 (m, 4 H, CH₂), 2.73-2.86 (m, 8 H, CH₂) inCH₂Cl₂-d₂ (−30° C.) | |
| 17 | 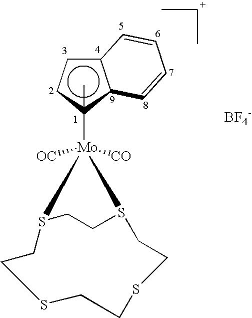 | | 3097 (w),1961 (vs, CO),1889 (vs, CO),1062 (vs,B—F),863 (w),763 (w) | 7.64 (m, 2 H, H^{5,8}), 7.54(m, 2 H, H^{6,7}), 6.29 (d,2 H, H^{1,3}), 5.32 (t, 1 H,H²), 3.48-2.67 (c, 16 H,H^{tetrathiociclododocane}) inCH₂Cl₂-d₂ | 506.9 (M^{+.},[IndMo(CO)₂(1,4,7,10-tetrt)]^{+.} |
| 18 | 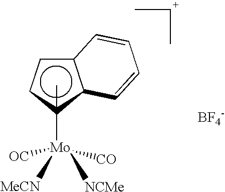 | C, 41.32; N, 6.42; H, 3.01(C, 41.0; N, 6.2; H, 2.91) | 3076, 2318,2290, 1970(vs, CO), 1892(vs, CO), 1062(vs) B—F) | | |
| 19 | 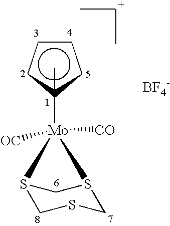 | C, 27.2; S, 21.8; H, 2.39(C, 27.2; S, 21.7; H, 2.51) | 1967 (vs,CO), 1893(vs, CO),1060 (vs,B—F) | 4.39 (s, 5 H, Cp), 3.71(br, 2 H, H⁶), 2.53 (br,4 H, H^{7,8}) in acetone-d₆ | |
| 20 | 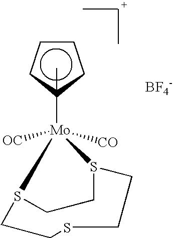 | C, 31.52; S, 19.45; H, 3.50(C, 32.25; S, 19.86; H,3.54) | 1967 (vs,CO), 1893(vs, CO),1060 (vs,B—F) | 5.72 (s, 5 H, Cp),3.88-2.45 (c, 12 H,H^{tten}) in CH₂Cl₂-d₂ | |
| 21 | 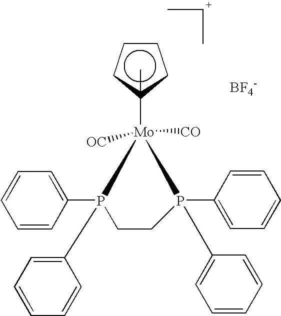 | | 1967 (vs,CO), 1893(vs, CO),1060 (vs,B—F) | 7.42-7.60 (m, 20 H,C₆H₅), 7.78 (s, 5 H,Cp), 1.55 (s, 4 H,CH₂); in CH₂Cl₂-d₂ | 617.0 (M^{+.},[CpMo(CO)₂dppe]^{+.} |
| 22 | 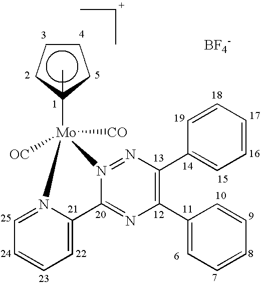 | C, 53.3; N, 9.76; H, 2.99(C, 52.8; N, 9.12; H, 3.12) | 1967 (vs,CO),1893 (vs,CO),1067 (vs,B—F),1368 (m) | 9.26 (d, 1 H, H²⁵),9.13 (d, 1 H, H^{14,19 or 6,10}),8.99 (d, 1 H,H²²), 8.75 (dd, 1 H,H²⁴), 8.25 (t, 1 H,H²³), 8.25, 7.89-7.41(c, 8 H, H^{6,10 or 14,}¹⁹⁺⁷⁻¹⁰⁺¹⁵⁻¹⁸),5.80 (s, 5 H,Cp) in CH₂Cl₂-d₂ | |
| 23 | 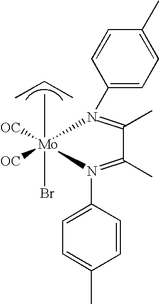 | C, 52.37; H, 5.36; N, 5.34(C, 51.41; H, 4.69; N, 5.21) | 1951; 1861 | | |
| 24 | 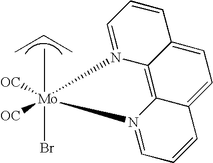 | C, 45.06; H, 2.89; N, 6.18(C, 44.69; H, 2.84; N, 6.05) | 1927; 1833 | | |
| 25 | 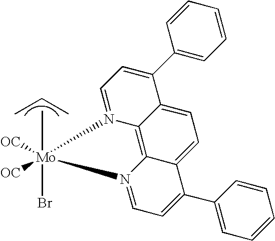 | C, 57.21; H, 3.68; N, 4.34.(C, 57.43, H, 3.4150;N, 4.62) | 1945; 1850 | | |

TABLE 2

| | | IC₅₀ value |
|---|---|---|
| Compd. | Structure | (μM) |
| (1) | 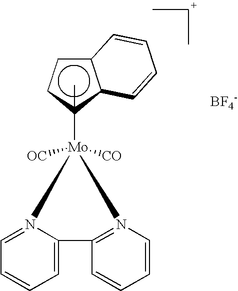 | 95.9 ± 1.1 |
| (2) | 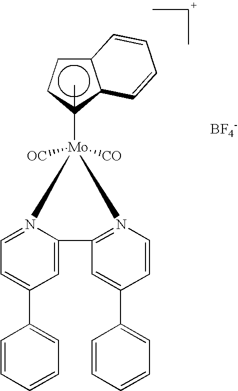 | 13.7 ± 1.1 |
| (3) | 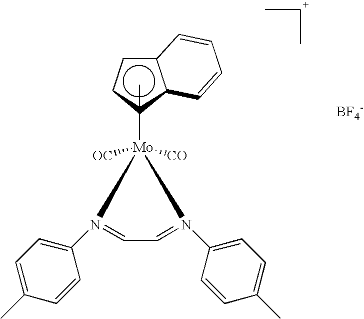 | 6.6 ± 1.0 |
| (4) | 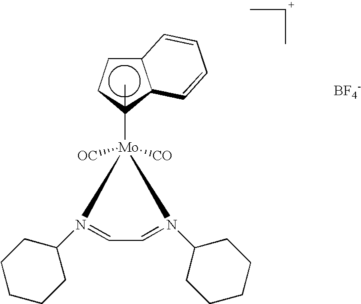 | 140.3 ± 1.2 |
| (5) | 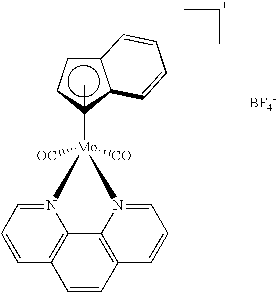 | 32.9 ± 1.0 |
| (6) | 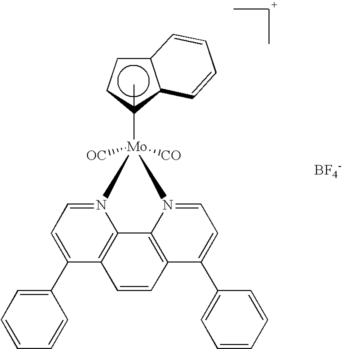 | 5.7 ± 1.0 |
| (7) | 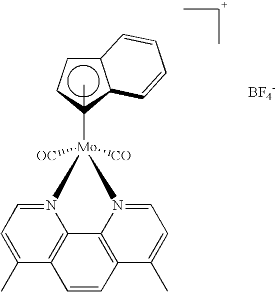 | 29.5 ± 1.0 |
| (8) | 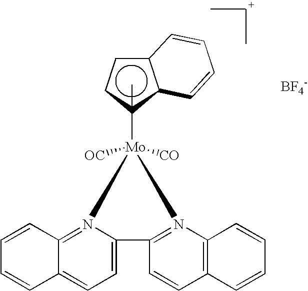 | 35.0 ± 1.1 |
| (9) | 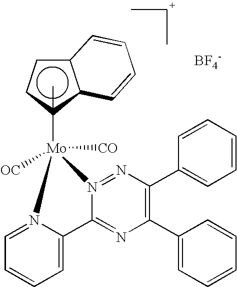 | 99.5 ± 1.1 |
| (13) | 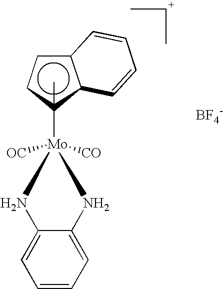 | 67.6 ± 1.2 |
| (14) | 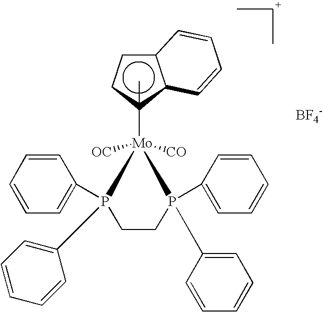 | 5.8 ± 1.1 |
| (15) | 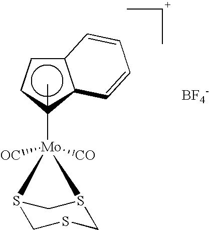 | 20.7 ± 1.0 |
| (16) | 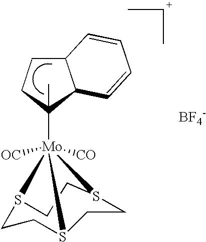 | 8.4 ± 1.1 |
| (17) | 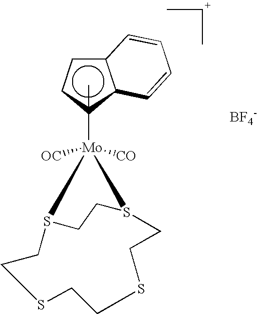 | 19.9 ± 1.1 |
| (18) | 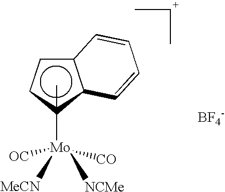 | 56.7 ± 1.7 |
| (19) | 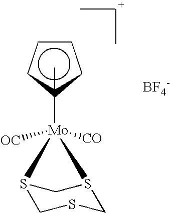 | 142.1 ± 1.1 |
| (20) | 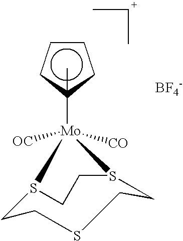 | 138.9 ± 1.2 |
| (21) | 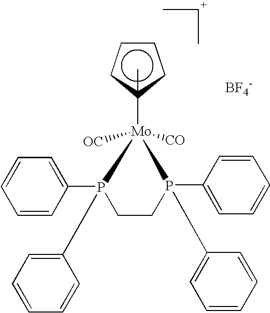 | 20.0 ± 1.1 |
| (22) | 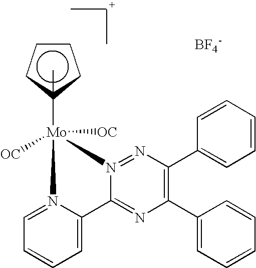 | 104.6 ± 1.2 |
| (23) | 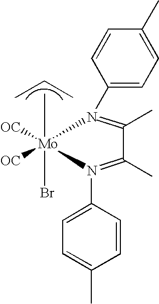 | 59.1 ± 1.1 |
| (24) | 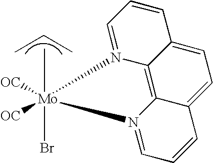 | 7.3 ± 1.0 |
| (25) | 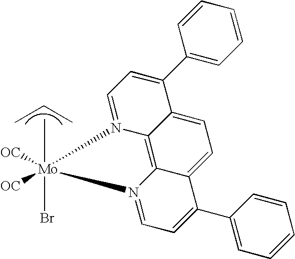 | 12.5 ± 1.2 |

TABLE 3

| | IC₅₀ VALUE (μM) | | | |
|---|---|---|---|---|
| | Human tumoral cell lines | | | |
| MOLYBDENUM (II) | Gastric | | | Colon |
| COMPLEX | GP-202 | GP-220 | MKN-45 | HT-29 |
| 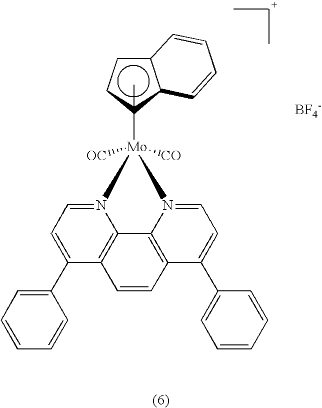 | 4.2 ± 1.1 | 1.8 ± 1.1 | 4.6 ± 1.1 | 6.6 ± 1.0 |
| 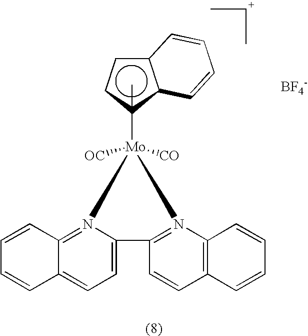 | 5.0 ± 1.1 | 2.7 ± 1.0 | 4.4 ± 1.0 | 13.3 ± 1.1 |

TABLE 4

| | | IC₅₀ value |
|---|---|---|
| Compd. | Structure | (μM) |
| (1) | 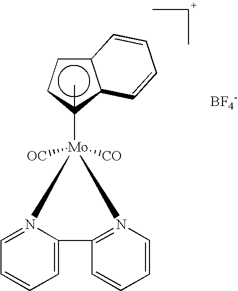 | 143.2 ± 1.1 |
| (3) | 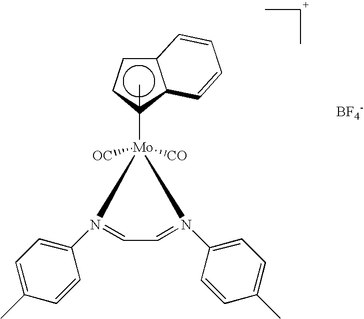 | 84.1 ± 1.0 |
| (4) | 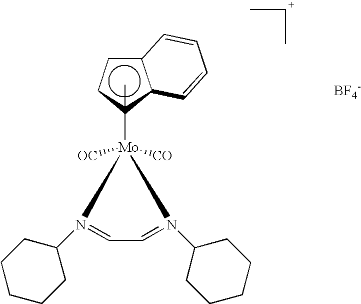 | 75.0 ± 1.1 |
| (5) | 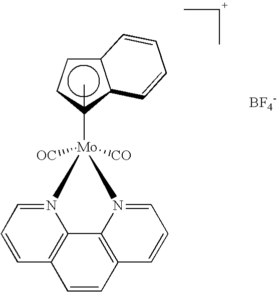 | 73.3 ± 1.1 |
| (6) | 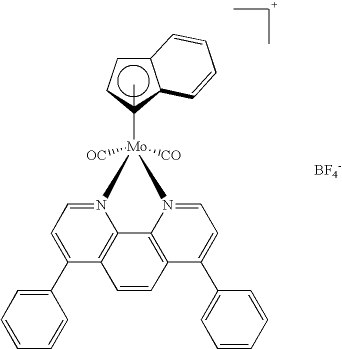 | 14.9 ± 1.0 |
| (7) | 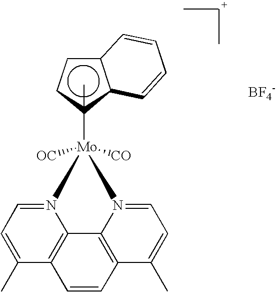 | 64.4 ± 1.1 |
| (8) | 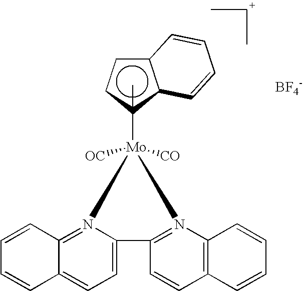 | 13.0 ± 1.0 |
| (9) | 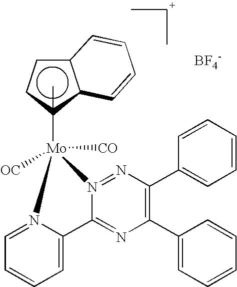 | 13.4 ± 1.0 |
| (13) | 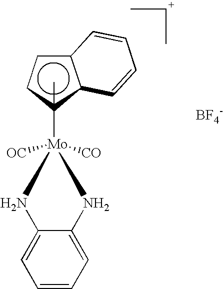 | 73.7 ± 1.1 |
| (14) | 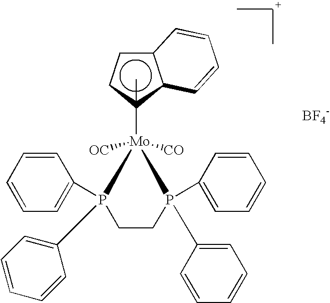 | 20.3 ± 1.1 |
| (15) | 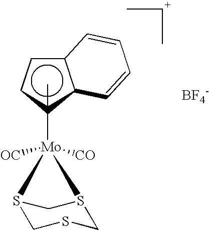 | 136.6 ± 1.0 |
| (16) | 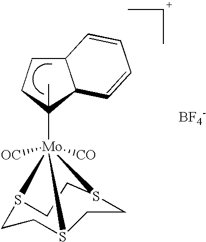 | 91.0 ± 1.1 |
| (17) | 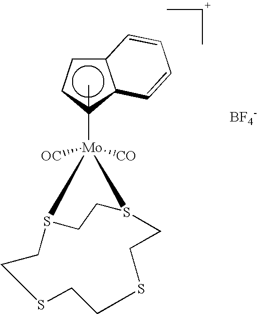 | 213.5 ± 1.1 |
| (20) | 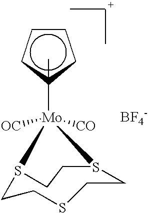 | 139.8 ± 1.2 |
| (21) | 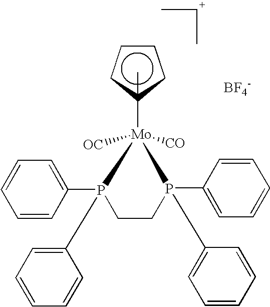 | 24.0 ± 1.0 |
| (22) | 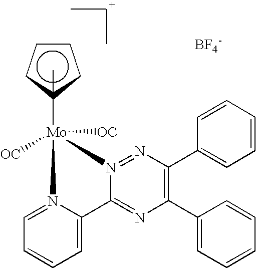 | 68.9 ± 1.1 |
| (23) | 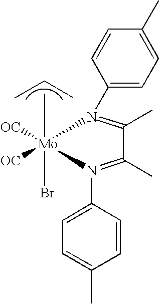 | 103.6 ± 1.1 |
| (25) | 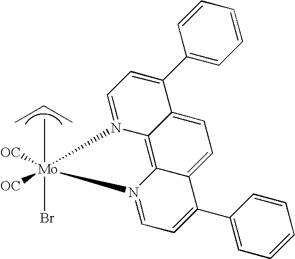 | 12.1 ± 1.1 |

The primary objectives of the present invention relate to medicinal agents having a cancerostatic effect characterized in that they contain at least one molybdenum complex with the general formula (I) or (II) (Figure I) as the active anticancer agent, in addition to pharmaceutically compatible vehicles, diluents and/or excipients and to the use of such agents in combating cancer.

The invention being described can be obviously varied in many ways. Such variations are not regarded as a departure from the spirit and scope of the invention, and all such modifications are intended to be included within the scope of the following claims.

What is claimed is:

1. A method for treating cancer in mammals consisting of a pharmaceutical composition, comprising an effective amount of an organometallic molybdenum (II) complex and a sterile non-toxic pharmaceutical acceptable vehicle therefor.

2. The method of claim 1 wherein, the organometallic molybdenum (II) complex is a compound of formula (I): 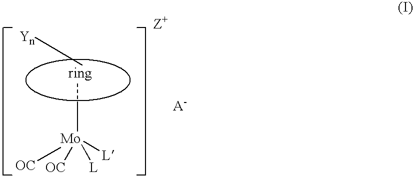
Wherein,
“ring” represents either cyclopentadienyl or indenyl;
Yₙ represents n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or one bidentate ligand with C, N, O, P or S donor atoms;
Z⁺ represents the overall charge of the Mo (II) complex, usually 1⁺ or 0;
A⁻ represents one suitable and pharmaceutically acceptable counter anion that equilibrate the complex charge when needed.

3. The method of claim 1 wherein, the molybdenum (II) complex is a compound of the general formula (II): 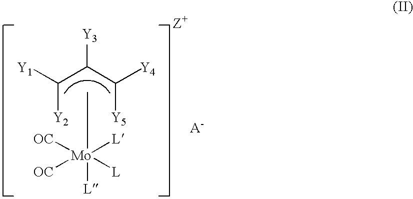
Wherein,
Y₁, Y₂, Y₃, Y₄, Y₅ represent n substituents which can be chosen, independently, from H, alkyl, alkenyl, alkoxy, aryl, halogen, haloalkyl, amino, organosilane (SiR₃), CO₂R, C(O)R, CHRCO₂R′, CHROH, cyano or nitro;
L and L′ represent either two independent monodentate ligands coordinated via C, N, O, P, S, halide donor atoms or one bidentate ligand with C, N, O, P or S donor atoms;
L″ represents one monodentate ligand coordinated via one C, N, O, P, S or halide donor atom;
Z⁺ represents the overall charge of the Mo (II) complex, usually 1⁺ or 0;
A⁻ represents one suitable and pharmaceutically acceptable counter anion that equilibrate the complex charge when needed.

4. A pharmaceutical composition according to claim 2 and 3 wherein, said pharmaceutical acceptable vehicle is selected from the group consisting of tablets, dragees, hard and soft gelatin capsules, dispersible powders and granules.

5. A pharmaceutical composition according to claim 2 and 3 wherein, said pharmaceutical acceptable vehicle is a physiological saline solution.

6. A pharmaceutical composition according to claim 2 and 3 wherein, said pharmaceutical acceptable vehicle is an isotonic sodium chloride solution.

7. A pharmaceutical composition according to claim 2 and 3 wherein, said pharmaceutical acceptable vehicle is an injectable vehicle.

8. A pharmaceutical composition according to claim 7 wherein, said injectable vehicle includes a physiological saline solution as the vehicle and dimethyl sulfoxide as a solubilizer.

9. A pharmaceutical composition according to claim 7 and further including a buffer.

10. A pharmaceutical composition according to claim 9, wherein, said buffer is sodium bicabornate or tris(hydroxymethyl)aminomethane.

11. A pharmaceutical composition according to claim 2 and 3 wherein, said pharmaceutical acceptable vehicle is an aqueous or oily suspension, emulsion, solution or syrup.

12. A liquid pharmaceutical composition according to claim 2 and 3 having pH of 4-7.

13. An injectable pharmaceutical composition according to claim 7 having a pH between 5.0 and 5.5.

14. A pharmaceutical composition according to claim 2 and 3 which contains an aqueous vehicle and a solubilizer.

15. A pharmaceutical composition according to claim 2 and 3 wherein, said composition is in the form of a suspension containing a liquid vehicle and a dispersing or wetting agent.

16. A pharmaceutical composition according to claim 2 and 3 wherein, said composition is in the form of an emulsion containing a liquid vehicle and an emulsifier.

17. A pharmaceutical composition according to claim 2 and 3 wherein, said composition is in the form of a water-dispersible powder or granule which contains said molybdenum (II) complex in a mixture with a dispersing, wetting or suspension agent.
